(19) United States
(12) Patent Application Publication
(10) Pub. No.: US 2026/0174946 A1
(43) Pub. Date: Jun. 25, 2026

(54) CONTINUOUS RIGHT ATRIAL PRESSURE MONITORING AND OCCLUSION

(71) Applicant: Edwards Lifesciences Corporation, Irvine, CA (US)

(72) Inventors: Allen Jeong Keel, Irvine, CA (US); Yaeer E. Lev, Torrance, CA (US); Atiya Makhdoom Ahmad, Irvine, CA (US); Daniel M. Harps, Trabuco Canyon, CA (US); Leonardo Paim Nicolau Da Costa, Tampa, FL (US)

(21) Appl. No.: 19/537,226

(22) Filed: Feb. 11, 2026

Related U.S. Application Data
(63) Continuation of application No. PCT/US2024/050789, filed on Oct. 10, 2024.
(60) Provisional application No. 63/591,696, filed on Oct. 19, 2023.

(51) Int. Cl. A61M 1/36; A61B 5/0215

(52) U.S. Cl. CPC A61M 1/3653; A61B 5/0215; A61B 5/02158

(57) ABSTRACT

Systems and methods are described for modulating blood flow through a blood vessel by providing an implantable device implanted in the blood vessel, monitoring, pressure in the blood vessel, and in response to detecting when a first pressure in the blood vessel is above a first predefined pressure range, causing actuation of the implantable device to modulate the blood flow through the blood vessel or an adjacent blood vessel according to a first actuation cycle configured to maintain the first pressure within the first predefined pressure range. In addition, the systems and methods may, in response to detecting, when a second pressure is above a second predefined pressure threshold, cause switching of the implantable device to a second actuation cycle to alter the modulation of the blood flow through the blood vessel.

### CROSS-REFERENCE TO RELATED APPLICATIONS

This application is a continuation of International Application No. PCT/US2024/050789, filed Oct. 10, 2024, which claims the benefit of U.S. Provisional Patent Application Ser. No. 63/591,696, filed Oct. 19, 2023, both of which applications are incorporated herein by reference in their entirety.

### BACKGROUND

This disclosure relates generally to the field of medical devices and procedures, and more specifically to the field of blood flow management in blood vessels.

When a patient is suffering from chronic Congestive Heart Failure (CHF), a clinician can measure right atrial pressure (RAP) values from a pulmonary artery catheterization to evaluate the congestion status of the patient. However, these RAP readings are limited to a hospital or clinic setting and generally provide a snapshot view of hemodynamic status at the time of the measurement. Once a patient exits the hospital or clinic setting, there is no further assessment for RAP until the patient returns the hospital or clinic to repeat the catheterization. This lack of visibility into understanding levels and excursions of RAP for a patient over time can lead to an incomplete understanding of blood volume status of the patient, which can lead to a suboptimal treatment and hospital reentry.

### SUMMARY

Described herein are one or more methods and/or devices to facilitate management and assessment of blood flow through and/or into one or more blood vessels and/or chambers of a heart. There is a need for new and useful system and method for monitoring blood flow in the venous system, modulating blood flow in the heart, and providing intermittent venous occlusion therapy.

In some aspects, the techniques described herein relate to an implantable device for modulating blood flow through a blood vessel and/or providing intermittent venous occlusion therapy to manage right atrial pressure and/or central venous pressure corresponding to intracranial venous pressure.

In some aspects, the techniques described herein relate to a method for modulating blood flow through a blood vessel, the method including: providing an implantable device implanted in the blood vessel; monitoring, by at least one processor, pressure in the blood vessel; in response to detecting, based on the monitoring, when a first pressure in the blood vessel is above a first predefined pressure range, causing actuation of the implantable device to modulate the blood flow through the blood vessel or an adjacent blood vessel according to a first actuation cycle of the implantable device configured to maintain the first pressure within the first predefined pressure range; in response to detecting, based on the monitoring, when a second pressure upstream of the first pressure is above a second predefined pressure threshold, causing switching of the implantable device to a second actuation cycle to alter the modulation of the blood flow through the blood vessel; and causing reversion of the implantable device to the first actuation cycle when the second pressure is detected to be within the second predefined pressure range.

In some aspects, the techniques described herein relate to an implantable device for dynamically modulating blood flow through a blood vessel, the implantable device including: an expandable frame including a proximal end and a distal end and a longitudinal axis extending therethrough; and an occlusion element including an inflow end and an outflow end, wherein the inflow end is at least partially installed within the distal end of the expandable frame, and the outflow end is coupled to an end effector configured to modulate the blood flow through the blood vessel; and a first sensor positioned at a distal end of the expandable frame and configured to detect a first pressure in a blood vessel; a second sensor positioned upstream of the first sensor and configured to detect a second pressure in the blood vessel at a location upstream of the first sensor; a processor electrically coupled to the first sensor and the second sensor, wherein the processor is configured to: monitor the first pressure and the second pressure sensed by the first and second sensors; and actuate the occlusion element to at least partially occlude the blood vessel at the end effector based on the monitored first pressure or the monitored second pressure.

In some aspects, the techniques described herein relate to a method for monitoring pressure in a subject in which a flow modulation device is implanted, the method including: in response to detecting a first pressure in a blood vessel, actuating the flow modulating device at least partially positioned in the blood vessel to a flow restriction state to at least partially restrict a flow of blood within the blood vessel or an adjacent blood vessel; continuously monitoring pressure at a location upstream of the detected first pressure in the blood vessel, the monitoring including: detecting a second pressure at the location; in response to determining that the second pressure is above a predefined pressure threshold, actuating the flow modulating device to a partially restricted state or an unrestricted state to at least partially release the restricted flow of blood; and in response to determining that the second pressure is at or below the predefined pressure threshold: maintaining the device in the flow restriction state until the second pressure is detected to exceed the predefined pressure threshold or upon completion of a predefined cycle configured for the flow modulating device; monitoring a rate of increase in the first pressure or monitoring a rate of increase in the second pressure; and actuating the flow modulating device based on the rate of increase in the first pressure or the rate of increase in the second pressure.

In some aspects, the techniques described herein relate to an implantable system for alleviating pressure within a blood vessel, the system including: a device for modulating a flow of blood through the blood vessel; a first sensor electrically coupled to the device and configured to detect a first pressure in a blood vessel; a second sensor electrically coupled to the device and configured to detect a second pressure in the blood vessel at a location upstream of the first sensor; a processing module electrically coupled to the first sensor and the second sensor, wherein the processing module is configured to: monitor outputs from the first sensor and the second sensor; and actuate the device to perform blood flow modulation through the blood vessel based on the monitoring of the outputs.

In some aspects, the techniques described herein relate to a method of treatment for reducing right atrial pressure for a first target region in a blood vessel of a heart of a subject and reducing intracranial pressure at a second target region associated with the subject, the method including: introducing a device in the blood vessel, the device including: a first sensor electrically coupled to the device and configured to detect a first pressure in a blood vessel; a second sensor electrically coupled to the device and configured to detect a second pressure in the blood vessel at a location upstream of the first sensor; a processing module electrically coupled to the first sensor and the second sensor and configured to monitor outputs from the first sensor and the second sensor; actuating, based on the monitoring of the outputs, the device to modulate a flow of blood within the blood vessel or an adjacent blood vessel.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates a bottom up perspective view of an example flow modulating device for monitoring pressure and modulating blood flow through a blood vessel.

FIG. 1B illustrates a side view of the example flow modulating device of FIG. 1A.

FIG. 1C illustrates a side view of the example flow modulating device of FIG. 1A including one or more potential stasis zones.

FIG. 1D illustrates a side view of the example flow modulating device of FIG. 1A including one or more stasis reducing features.

FIG. 2 is an example system for monitoring and reducing pressures in a blood vessel.

FIG. 3 is an example system for managing time-in-range right atrial pressure for a subject.

FIG. 4 is an example system for maximizing time-in-range right atrial pressure while minimizing a time in which the superior vena cava time exhibits increased pressure.

FIG. 5 is an example graph depicting an example cycle of blood flow modulation using an implanted flow modulating device.

FIG. 6A is a flow diagram depicting an example process for assessing and modulating pressures in a blood vessel.

FIG. 6B is an example table representing a number of parameters that may be tuned to modify operation of the devices described herein.

FIG. 7A is a flow diagram depicting an example process for monitoring and modulating pressures in a blood vessel.

FIG. 7B is an example table representing a number of parameters that may be tuned to modify operation of the devices described herein.

FIG. 8 illustrates a block diagram of an example system for modulating blood flow through one or more blood vessels.

FIG. 9 illustrates a schematic diagram of an example embodiment of a system for modulating blood flow through a blood vessel.

FIG. 10 illustrates a schematic diagram of an example embodiment of a system for modulating blood flow through a blood vessel.

FIG. 11 is a flow diagram of an example process for modulating blood flow through one or more blood vessels.

FIG. 12 illustrates a flow diagram of an example process of modulating blood flow through one or more blood vessels.

FIG. 13 illustrates a schematic representation of portions of a human subject in which the devices described herein may be implanted.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiments(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

In general, the systems and methods described herein may enable monitoring, modulating and/or balancing of blood flow through a blood vessel to maintain the stability of the pressure in the heart and/or other organs. The modulating and/or balancing of blood flow may be performed by the devices described herein to occlude, partially occlude, and/or otherwise manage or regulate blood flow to or through a portion of a blood vessel. In some examples, such modulation and/or balancing of blood flow to or through a blood vessel may result in additionally modulating pressure in the right atrium of the heart and/or other organs of the body. In addition, the systems and methods described herein may perform real time (or near real time) pressure analysis for a subject (e.g., a patient) in order to generate metrics that provide an assessment indicating a level of congestion risk for the subject. The pressure analysis may be performed on data obtained by monitoring pressure in a blood vessel at one or more sites in the blood vessel. The monitoring can be performed to inform the devices described herein with instructions for how to occlude, partially occlude, and/or otherwise manage or regulate blood flow to or through a portion of a blood vessel(s).

The examples presented herein may relate to providing devices, methods, and/or methods of treatment (MOTs) for modulating, regulating and/or otherwise managing blood flow to or through one or more blood vessels. The terminology of restricting blood flow, regulating blood flow, modulating blood flow, managing blood flow, and balancing blood flow cause regulation of blood pressure, modulation of blood pressure, management of blood pressure, and/or balancing of blood pressure. As such, for example, a flow modulation device is synonymous with a pressure regulating device (i.e., a flow regulator is synonymous with a pressure regulator). In some examples, the devices described herein may include blood flow management devices for reducing blood flow through a blood vessel, such as the Vena Cava (VC), the Superior Vena Cave (SVC), the Inferior Vena Cava (IVC), or related vessels. Managing blood flow through the VC, SVC or IVC can be achieved by the devices described herein to provide an advantage of improving perfusion of the kidneys. In particular, the devices described herein may generate a pressure gradient across the kidneys by decreasing central venous pressure by restricting, balancing, or otherwise modifying blood flow through the VC, SVC and/or IVC, resulting in improved kidney perfusion and function. Managing blood flow through the VC, SVC, or IVC can be achieved by the devices described herein to provide an advantage of improving upstream SVC pressure that impacts central venous pressure and/or intracranial venous pressure.

In some examples, the devices, methods, and/or MOTs described herein may be utilized to solve a technical problem of detecting unwanted pressure increases in one or both of the right atrium (i.e., right atrial pressure (RAP)) and upstream pressure in the SVC in subjects that have chronic kidney disease (CKD) and/or heart failure (HF). For example, subjects with CKD and/or HF may exhibit reduced kidney function when pressure in the right atrium of the heart is above a predefined pressure threshold. The predefined pressure threshold may be used as a basis to determine whether a subject is exhibiting low vessel pressure (e.g., below the predefined pressure threshold) or high vessel pressure (e.g., above the predefined pressure threshold). When vessel pressure is determined to be high, the devices, methods, and/or MOTs can provide a technical solution to the technical problem recited above by monitoring and actively modulating blood flow through a blood vessel responsive to the monitoring. In addition, vessel pressure upstream of an occlusion site of an implanted device (e.g., in the SVC) may be detected and utilized in combination with detected pressure at or near to the occlusion site to reduce or alleviate both RAP and upstream SVC pressure. For example, the devices described herein may be used to decrease pressure within one or more vessels to avoid right atrium pressure increases and/or pressure variations while monitoring SVC pressure and modifying operation of the device to further decrease SVC pressure when such pressure begins to elevate. In particular, the devices, methods, and/or MOTs described herein can be used to reduce and maintain low pressure in the right atrium and/or low pressure in the SVC (e.g., low intracranial pressure) which provides a technical effect of enabling the kidneys to improve an efficiency and/or effectivity when filtering blood.

In addition, the devices, methods, and/or MOTs described herein can solve a further technical problem of capturing pressure measurements multiple times a day (e.g., intermittent capture throughout a day, capture on a schedule throughout a day, continuous capture, near continuous capture, etc.). Conventional implantable cardiac pressure sensor technologies that capture and transmit intracardiac pressure data are limited to once daily (e.g., static) measurements due to reliance on an external unit to power the sensor with RF energy. These types of conventional snapshot measurement systems lack the temporal resolution to capture the data about how much total time a subject is spending within (or outside of) an optimal pressure range for RAP and/or SVC pressure. The devices, methods, and/or MOTs described herein can use one or more sensors to detect RAP and/or SVC pressures and determine a time-in-range RAP metric (determined using analysis of one or more sensor outputs). The time-in-range RAP metric may provide an assessment of real time congestion risk for subjects with HF. The sensor outputs may be captured over time and assessments may be performed in an ambulatory setting or in a clinic or hospital setting.

In some examples, the detected/monitored pressures obtained by the devices described herein may function as a feedback mechanism to maintain a predefined pressure in the venous system. For example, monitoring an upstream SVC pressure may be used as feedback for operating an occlusion device attempting to maintain a particular RAP pressure (or pressure range). Having such a feedback mechanism that collects upstream SVC pressure in conjunction with RAP (e.g., pressure downstream of the SVC pressure or downstream of the device) may allow the device to control occlusion in a manner to optimize the RAP reduction benefit while minimizing the time that the upstream/SVC pressure is in an unsafe range.

In operation, the devices described herein may be used to dynamically monitor and reduce the accumulation of blood in the venous system, which can provide an advantage and technical effect of ensuring that pressure is not increased in the SVC, the cranium, and/or the IVC. Such devices can advantageously eliminate excessive hospital readmissions and/or can provide for a long-term blood flow management therapy, improving both quality of life and overall survival rates and with a lower cost to a healthcare system.

Maintaining a particular time-in-range RAP metric by dynamically monitoring one or more sites within a blood vessel may ensure that a subject spends a higher percentage of time within a safe RAP range. A higher percentage of time-in-range RAP may be an indicator that a left atrial pressure (LAP) of a subject is in a controlled state and that renal venous pressure is lower, indicating unloaded kidneys that are more likely to function to diurese effectively.

The time-in-range RAP metric may be based on output(s) captured by one or more sensors onboard the occlusion devices described herein. The sensors may be battery-powered implantable pressure sensors which can sample pressures frequently, offering continuous or near-continuous measurement of RAP. An onboard processor enables a computation of time-in-range RAP by taking programmable inputs defining one or more thresholds for which pressure values are considered in range (e.g., healthy pressures), as described elsewhere herein.

Furthermore, the devices, methods, and/or MOTs described herein can be used to solve a further technical problem of regulating (e.g., modulating) blood flow return, thus further mitigating pressure build-up in the right atrium and SVC. The examples described herein can perform blood flow management actively and/or passively to assist in reducing and/or maintaining right atrium pressures to a relatively low pressure even when a surge in blood volume occurs in one or more vessels of the venous system. In some examples, such blood flow management may be performed based on monitoring pressures using one or more sensors onboard the occlusion device and algorithms executing on an onboard processor as described in detail elsewhere herein. In some examples, the monitoring may be performed on an external computing device in communication with the occlusion device and/or sensors of the occlusion device.

In some examples, the devices, methods, and/or MOTs described herein can be used to trigger and perform one or more agitation cycles for reducing blood stasis through and near to a blood vessel site in which the occluding device is implanted. Example agitation cycles may be executed for a time frame of about 5 seconds to about 10 minutes. For example, an agitation may be executed using the devices described herein for: about 5 seconds to about 30 seconds; about 20 seconds to about 40 seconds, about 30 seconds to about 40 seconds; about 40 seconds to about 1 minute; about 1 minute to about 2 minutes; about 2 minutes to about 5 minutes; about 4 minutes to about 6 minutes; about 5 minutes to about 7 minutes; about 6 minutes to about 8 minutes; about 7 minutes to about 9 minutes; about 8 minutes to about 10 minutes.

In some examples, the agitation cycle may begin by opening an end effector at a rate of about 0.1 seconds to about 1 second and may close an end effector at a rate of about 0.1 seconds to about 1 second. In some examples, the agitation cycle may begin by opening at a rate of about 1 minute to about 5 minutes; about 1 minute to about 2 minutes; about 2 minutes to about 3 minutes; about 3 minutes to about 4 minutes; or about 4 minutes to about 5 minutes. In some examples, the agitation cycle may close an end effector at a rate of about 1 minute to about 5 minutes; about 1 minute to about 2 minutes; about 2 minutes to about 3 minutes; about 3 minutes to about 4 minutes; or about 4 minutes to about 5 minutes. The agitation cycle may be performed in a fully open or a partially open state.

### Systems and Devices

Disclosed herein are systems and methods for modulating blood flow through a blood vessel and monitoring pressures within the blood vessel. In some examples, the implantable flow modulating devices described herein may be used in blood flow occlusion therapy. For example, the devices described herein may relate to venous occlusion therapy using implantable mechanically, hydraulically, and/or electronically controlled flow restricting devices for the treatment of acute heart failure. Some devices may be non-implantable or partially implantable. In some examples, the devices described herein generally function to occlude or partially occlude a blood vessel, such as the SVC or the IVC. In some examples, the devices described herein have been contemplated for use in a subject having chronic heart failure and/or chronic kidney disease, but may be used in any vessel needing flow regulation therethrough.

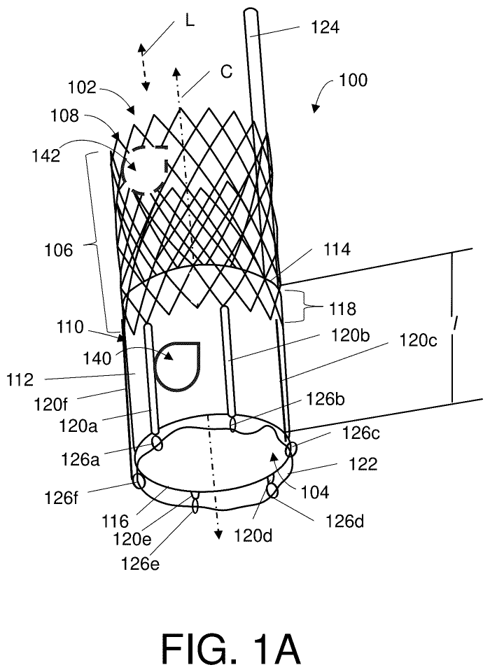

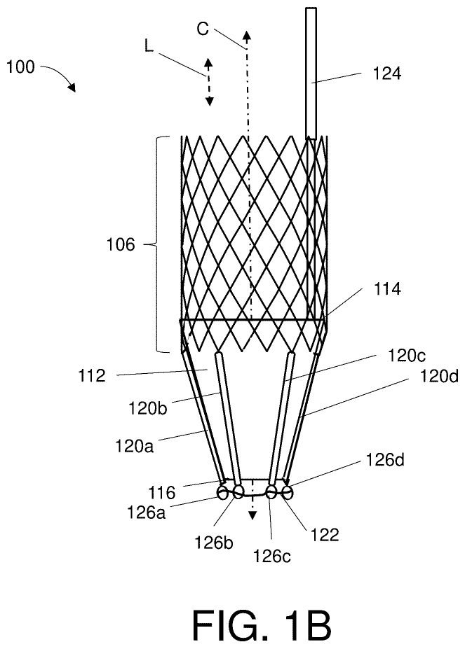

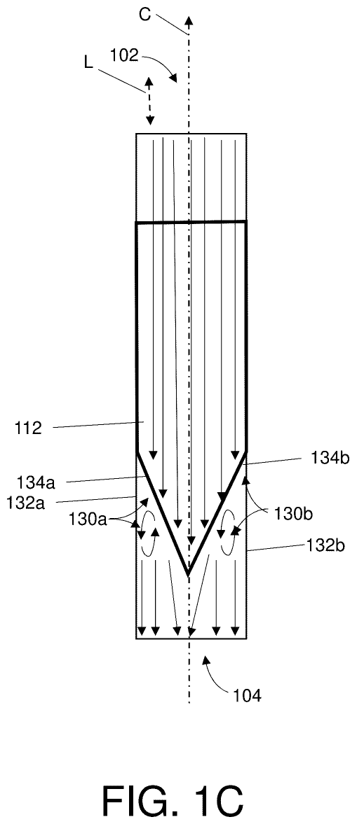

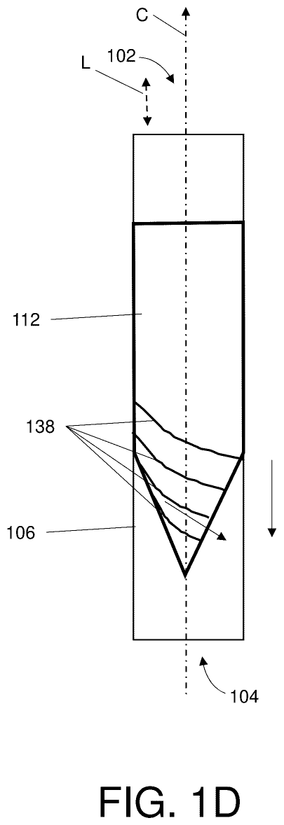

FIGS. 1A-1D illustrate views of an example flow modulating device 100 for monitoring pressure and modulating blood flow through a blood vessel. The device 100 may be implanted into a blood vessel, such as the SVC, the IVC, or any other blood vessel where modulating blood flow is desired. For example, the device 100 may be implanted in the SVC above a junction between the SVC and the right atrium. The device 100 may modulate a volume of blood flowing from the superior vena cava into a right atrium to decrease right atrial pressure and/or decrease SVC pressure and thus reduce intracranial venous pressure.

At a high level, the device 100 may include a self-expanding or balloon expandable frame (e.g., stent) that may be delivered into the blood vessel (e.g., via jugular access, subclavian access, or transfemoral access) using a sheathed catheter (not shown). The frame may include or be coupled to an occlusion element (e.g., a membrane) that is further coupled to a flexible control wire threaded through a portion (e.g., end effector) of the membrane. The flexible control wire may function as a lasso to be actuated by an actuation device to radially expand and constrict (uniformly or nonuniformly) a perimeter of the end portion of the membrane to function as an adjustable blood flow restrictor.

FIG. 1A illustrates a bottom up perspective view of an example flow modulating device 100 for monitoring and modulating blood flow through a blood vessel. In this example, the device 100 is shown in an unrestricted blood flow state. The unrestricted blood flow state may represent a state of device 100 in which both an inflow 102 and an outflow 104 are open to receive fluid (e.g., blood, drugs, saline, etc.). The fluid flows through the inflow 102 and through the device 100 to the outflow 104. For example, the device 100 may be in the expanded state when both the inflow 102 and the outflow 104 are open to receive fluid (e.g., blood, drugs, saline, etc.) therethrough when the device 100 is implanted in a blood vessel.

The device 100 includes an expandable frame 106 that includes a proximal end 108 and a distal end 110, and a longitudinal axis (L) extending therethrough. The proximal end 108 may correspond to the inflow 102 of the device 100. The frame 106 may be a stent, for example constructed of metal wire (e.g., stainless steel, platinum, Nitinol® wire or another shape memory alloy), or other material suitable for implantation in the human body. In some examples, the expandable frame 106 is a bare metal stent, such that the expandable frame 106 is configured to be at least partially incorporated into an inner wall of the blood vessel. In some examples, the expandable frame 106 has a pro-endothelialization coating, such that the expandable frame can be at least partially incorporated into an inner wall of the blood vessel. This incorporation may allow a site of the device 100 to maintain a non-thrombogenic, non-immunogenic environment with respect to the device 100. For example, the coating of the frame 106 may be any pro-endothelial factor including, but not limited to, endothelial growth factor, vascular endothelial growth factor, or any related compound.

The device 100 also includes a membrane 112 with an inflow end 114 and an outflow end 116. The inflow end 114 is shown at least partially installed within the distal end 110 of the expandable frame. For example, the membrane 112 is coupled to an inner surface portion of the expandable frame 106, as shown by an overlap 118. The membrane 112 may be installed within (and overlapping) about 25% of a length of the frame 106. In some examples, the membrane 112 may be installed within (and overlapping) about 10% to about 50% of the length of the frame 106. In some examples, the membrane 112 may be installed within (and overlapping) about 10% to about 25% of the length of the frame 106. In some examples, the membrane 112 may be installed within (and overlapping) about 20% to about 30% of the length of the frame 106. In some examples, the inflow end 114 of the membrane 112 is decoupled from the distal end 110 of the frame 106 and without an overlap. For example, the inflow end 114 may be reversibly coupled to the distal end 110 of the frame 106. The membrane 112 may be formed of a polymer a copolymer, a textile (e.g., woven, knitted, nonwoven, or braided), a tissue (e.g., bovine pericardium, equine pericardium, porcine vena cava, etc.), or a combination thereof.

In some examples, the membrane 112 is substantially tubular-shaped with a substantially circular cross section about a central axis (C). In some examples, the membrane 112 may be substantially elliptical in shape with a substantially elliptical cross section about the central axis (C). In some examples, the membrane 112 may be substantially flexible such that the shape may take on an irregular perimeter that may form a shape of the blood vessel in which the device 100 is installed, for example, when blood flow is provided from the inflow end 114 through to the outflow end 116.

In some examples, the membrane 112 is adjustable to any number of positions between expanded and collapsed. The membrane 112 may be adjustable to form a cinched portion at the outflow end 116. The cinching may result in reversibly reducing or closing the circular cross section at the outflow end 116 of the membrane 112. For example, the outflow end 116 may collapse inward toward the central axis (C) associated with the frame 106 and at any interval between fully expanded and fully contracted. The outflow end 116 may also open or expand outward away from the central axis (C) associated with the frame 106. In some examples, the membrane 112 may be expanded or contracted from a particular device state into an expanded position, a partially expanded position, or a collapsed position. For example, when the device 100 is in the expanded position, the device 100 may be caused to be configured into a partially expanded position or a collapsed position by partially or fully collapsing, respectively, the outflow end 116 of the membrane 112 toward the central axis (C). When the device 100 is in the collapsed position, the device 100 may be caused to be configured into a partially expanded position or an expanded position by partially or fully expanding, respectively, the outflow end 116 of the membrane 112 toward the central axis (C).

The expanded position of the membrane 112 may allow the blood flow through the blood vessel. For example, when the device 100 is implanted in a blood vessel and is configured in the expanded position, the device 100 may allow blood to flow from the inflow end 114 through to the outflow end 116, without substantially hindering the blood flow speed or the blood flow amount.

The partially expanded position of the membrane 112 may allow partial occlusion of the blood vessel. For example, when the device 100 is implanted in a blood vessel and is configured in the partially expanded position, the device 100 may allow a partial amount of blood to flow from the inflow end 114 through to the outflow end 116 and may hinder a flow of the blood flow by a predefined amount associated with a cross sectional area formed when the outflow end 116 is partially closed (e.g., partially collapsed, partially expanded).

The collapsed position of the membrane 112 may occlude the blood vessel. In some examples, the occlusion of the blood vessel is a full occlusion. In some examples, the occlusion of the blood vessel is a partial occlusion.

As shown in FIG. 1A, the outflow end 116 of the membrane 112 is coupled to a plurality of elongate support members 120a, 120b, 120c, 120d, 120e, and 120f. The elongate support members 120a-120f may be flexible to allow the membrane 112 to bend radially toward the central axis (C) of the frame 106 at the outflow end of the membrane 112 when the control wire is actuated.

In some embodiments, actuating the membrane 112 is caused by actuation of the control wire coupled to a portion of the membrane 112) or at least one of the elongate support members 120a-120f For example, actuating the control wire may result in configuring the membrane 112 in an unrestricted blood flow state or a restricted blood flow state. The unrestricted blood flow state may correspond to the membrane 112 radially expanding away from the central axis (C) of the expandable frame 106 to allow blood flow through the blood vessel. The restricted blood flow state may correspond to the membrane 112 radially collapsing toward the central axis (C) of the expandable frame to reduce blood flow through the blood vessel.

The plurality of support members 120a-120f may be arranged radially around the membrane 112. For example, the plurality of support members 120a-120f may be arranged radially around an outer surface or an inner surface of the membrane 112. For example, the plurality of elongate support members 120a-120f may be equidistantly arranged radially around a surface of the membrane 112. In some examples, the plurality of elongate support members 120a-120f may be arranged non-equidistantly around a surface of the membrane 112. In some examples, the plurality of elongate support members 120a-120f may be arranged radially around a surface of the membrane 112 such that support members 120a-120c are arranged around a first semi-circular and surface portion of the membrane 112 while support members 120d-120f are arranged around a second semi-circular portion of the membrane. For example, the support members 120a, 102b, and 120c may be separated by substantially similar distance apart around the first semi-circular and surface portion of the membrane 112 and the support members 120d, 102e, and 120f may be separated by substantially equidistant apart around the second semi-circular and surface portion of the membrane 112. In such an arrangement, the support member 120c may be arranged adjacent to support member 120d, but may be arranged at a closer distance than the distance between support member 120a and 120b or between support member 120b and support member 120c. Similarly, the support member 120a may be arranged adjacent to support member 120f, but may be arranged at a closer distance than the distance between support member 120c and 120d or between support member 120e and support member 120c.

In some examples, the plurality of elongate support members 120a-120f may extend from the outflow end 116 and toward the inflow end 114 running substantially parallel to the longitudinal axis (L) of the device 100. The support members 120a-120f may extend a portion of a length (l) of the membrane 112. For example, the support members 120a-120f may extend across about 50% to about 90% of an outer surface of the membrane 112. In some examples, the support members 120a-120f may extend a full length (l) of the membrane 112. The length of the membrane 112 may be about 5 millimeters to about 5 centimeters. The radius of the membrane 112 may be about 10 millimeters to about 30 millimeters. The thickness of the membrane 112 may be about 0.01 millimeters to about 1 millimeter.

Although the device 100 includes six support members 120a-120f, more or fewer support members are possible. For example, the device 100 may have three to five support members; four to six support members; five to seven support members; or five to eight support members.

As shown in FIG. 1A, the device 100 further includes an eyelet 126a, an eyelet 126b, an eyelet 126c, an eyelet 126d, an eyelet 126e, and an eyelet 126f. The eyelets 126a-126f may be coupled to respective support members 120a-120f For example, the eyelet 126a is coupled to a distal end of the support member 120a; the eyelet 126b is coupled to a distal end of the support member 120b; the eyelet 126c is coupled to a distal end of the support member 120c; the eyelet 126d is coupled to a distal end of the support member 120d; the eyelet 126e is coupled to a distal end of the support member 120e; the eyelet 126f is coupled to a distal end of the support member 120f. Each eyelet 126a-126f may be configured to receive a portion of the control wire 122 threaded therethrough. The eyelets 126a-126f extend beyond the distal end of each respective support member 120a-120e. Each eyelet 126a-126f is formed as an aperture having a substantially annular opening. The aperture of each respective eyelet 126a-126e is arranged to receive the control wire 122 when threaded therethrough such that when the control wire 122 is actuated, the eyelets 126a-126f move radially (e.g., cinching each eyelet together) toward the central axis (C) of the expandable frame 106. For example, actuating the control wire 122 reversibly cinches the membrane 112 toward the central axis (C) by bringing the eyelets 126a-126f together at the outflow end 116 of the membrane 112 to occlude or partially occlude a blood vessel in which the device 100 is implanted.

Referring again to FIG. 1A, the outflow end 116 of the membrane 112 may correspond to the outflow 104 of device 100. The outflow end 116 of the membrane 112 may be triggered to radially collapse toward the central axis (C) associated with the frame 106. For example, the outflow end 116 of the membrane 112 may be configured to radially collapse inward at the outflow end 116 by moving the plurality of support members 120a-120f and attached eyelets 126a-126f toward the central axis (C) or radially collapse outward at the outflow end 116 by moving the plurality of support members 120a-120f and attached eyelets 126a-126f away from the central axis (C). The radial collapse or expansion may occur in response to an actuation of a control wire 122. The control wire 122 may be coupled to a portion of the membrane 112 or at least one of the elongate support members 120a-120f to trigger the expansion or the collapse.

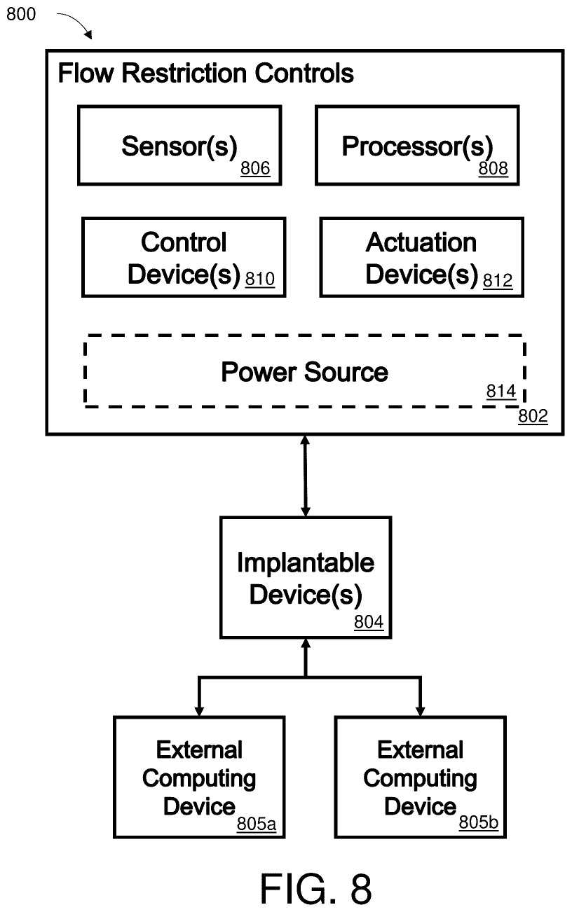

In some examples, the device 100 may further include a first sensor 140 (e.g., in sensors 806 of FIG. 8) for detecting a pressure (e.g., RAP) in the blood vessel at a first location. The device 100 may also include an optional second sensor 142 (e.g., in sensors 806 of FIG. 8) for detecting a pressure (e.g., SVC pressure) in the blood vessel at a second location upstream from the first location. The output from sensor 140 and/or sensor 142 may be used to sample one or more pressures frequently to provide a continuous or near continuous measurement of RAP and a feedback mechanism for adjusting the device 100 to occlude more or occlude less based on the sensor output. In general, the sensor 140 and sensor 142 may represent implantable pressure sensors that enable capture and transmission of pressure data for use in dynamically monitoring and dynamically occluding intravascular devices.

In some examples, the device 100 includes one or more processors (e.g., processor 808) electrically coupled to one or more sensors (e.g., sensor 140, optional sensor 142, sensors 806, etc.), and/or a power source (e.g., power source 814) electrically coupled to an actuator (e.g., actuation device 812) associated with device 100, the processor 808, and/or the sensors described herein. For example, the sensor 140 and/or the optional sensor 142 may sense characteristics of blood flow in the blood vessel (e.g., blood pressure, patterns of blood pressure, patient state, etc.) and may cause the processor to provide signals to the actuator (e.g., actuation device 812) and/or control element 124 and/or control wire 122 (e.g., control devices 810). In operation, the processor 808 can receive a signal from the sensor 806 that is indicative of a pressure in the blood vessel. The processor 808 can process the signal and generate and provide a control signal (e.g., via control devices 810) to tension the control devices 810, or release tension in the control devices 810 based on the sensed pressure in the blood vessel. The tensioning and release of tension may be performed based on monitoring performed by the processor while utilizing sensor 140 and/or optional sensor 142.

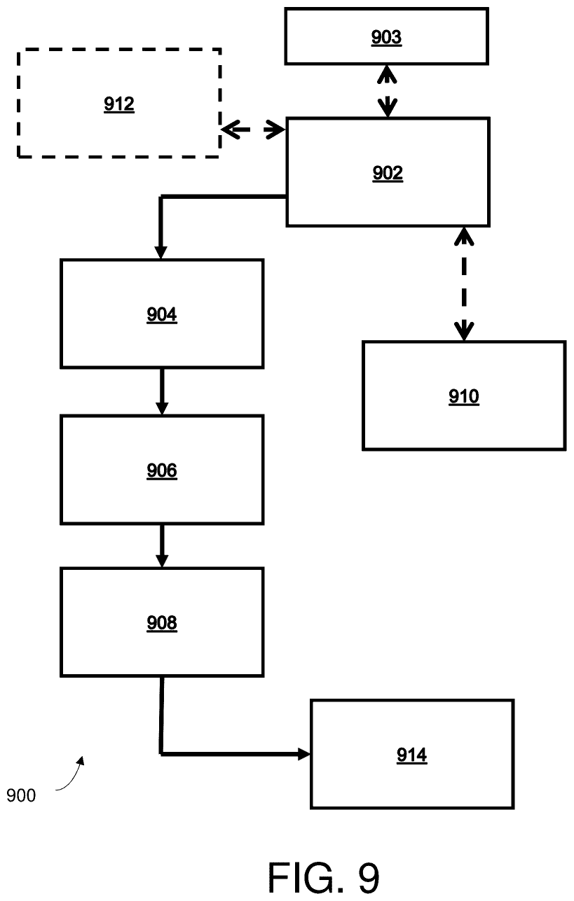

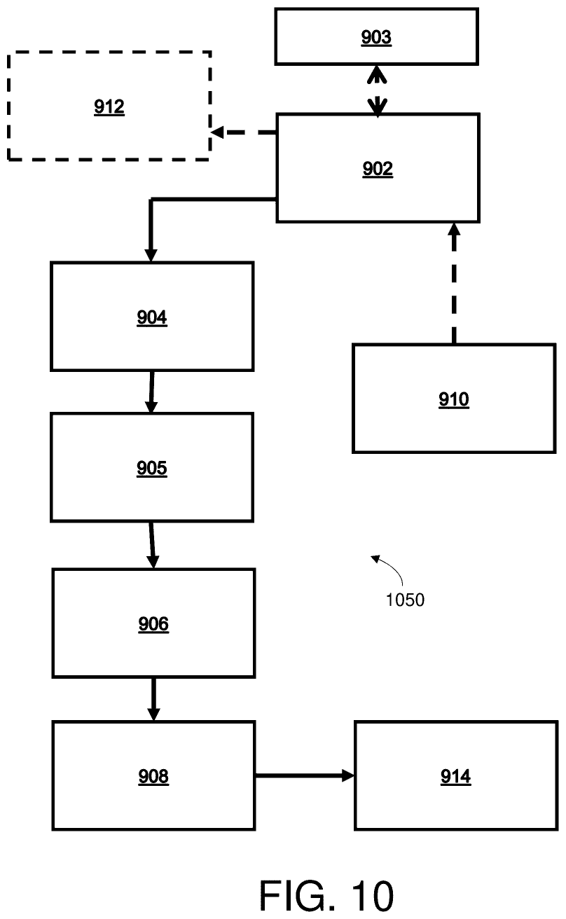

In some examples, the sensor 140 and/or optional sensor 142 may be communicatively coupled to device 100. The sensor 140 may include one or more of an image sensor, a strain gauge, a piezoelectric sensor, a fiberoptic sensor, a capacitance sensor, and/or a vacuum pressure sensor. The optional sensor 142 may include one or more of an image sensor, a strain gauge, a piezoelectric sensor, a capacitance sensor, and/or a vacuum pressure sensor. If the device 100 is coupled to a power source (e.g., power source 814), the power source may include an induction coil. The induction coil may be used to operate one or more of such magnets, as described in further detail in FIG. 9 and/or FIG. 10.

In some examples, the device 100 is an implantable device for dynamically modulating blood flow through a blood vessel such as the SVC. For example, the device 100 may be implanted in a subject and may modulate a volume of blood flowing from the SVC into a right atrium to decrease right atrial pressure and/or to modulate intracranial venous pressure according to rules and/or parameters programmed into the device 100.

In some examples, the device 100 is an implantable device for dynamically modulating blood flow through a blood vessel such as the IVC. For example, the device 100 may be implanted in a subject and may modulate a volume of blood flowing from the IVC to decrease venous pressure and/or to modulate intracranial venous pressure according to rules and/or parameters programmed into the device 100.

In some examples, the device 100 may include an expandable frame 106 including a proximal end 108, a distal end 110, and a longitudinal axis extending therethrough. The device 100 may also include a membrane 112 with an inflow end 114 and an outflow end 116. The inflow end 114 may be at least partially installed within the distal end 110 of the expandable frame 106. The outflow end 116 may be coupled to a plurality of elongate support members 120a-120f arranged radially around an outer surface of the membrane 112 and extending substantially parallel to the longitudinal axis (L). The device 100 may further include a first sensor 140 positioned at or adjacent to a distal end 110 of the expandable frame. The first sensor 140 may detect a first pressure in a blood vessel. The device 100 may optionally include a second sensor 142 positioned upstream of the first sensor 140 toward a proximal end 108 of the expandable frame 106. The optional second sensor 142 may detect a second pressure in the blood vessel at a location upstream of the first sensor 140.

In some examples, the device 100 may include one or more processors (e.g., processor 808, processor 903, etc.). The processor may be electrically coupled to the first sensor 140 and the optional second sensor 142. The processor may be programmed to carry out instructions for monitoring the first pressure and the second pressure via the sensors, for example. The processor may be further programmed to carry out instructions for actuating the membrane 112 to at least partially occlude the blood vessel based on the monitoring (e.g., the monitored first pressure or the monitored second pressure as detected by respective sensors 140, 142).

In some examples, actuating the membrane reduces blood flow through the blood vessel to reduce the first pressure and reduce the second pressure. For example, the first pressure may represent RAP and the second pressure may represent SVC pressure upstream of the right atrial pressure. The membrane 112 may radially collapse at the outflow end 116 and toward a central axis (C) of the expandable frame 106 to reduce the RAP. The radial collapse of the membrane 112 may be based on any one or more of predefined occlusion cycles, detected pressure ranges, occlusion rules, and parameter settings, etc.

In some examples, actuating the membrane increases blood flow through the blood vessel to reduce at least a portion of the second pressure. For example, the membrane 112 may radially expand at least a portion of the membrane 112 away from the central axis (C) of the expandable frame 106 to alleviate occlusion. In such an example, the device 100 may be triggered to release the occlusion by a particular percentage in response to detecting pressure upstream of the occlusion site. In some examples, the device 100 may be triggered to release the occlusion by a particular percentage in response to reaching the end of a predefined occlusion cycle. The device 100 may be triggered to release the occlusion by a particular percentage in response to other rules or parameters that may be configured for the device 100 and/or a particular component of device 100.

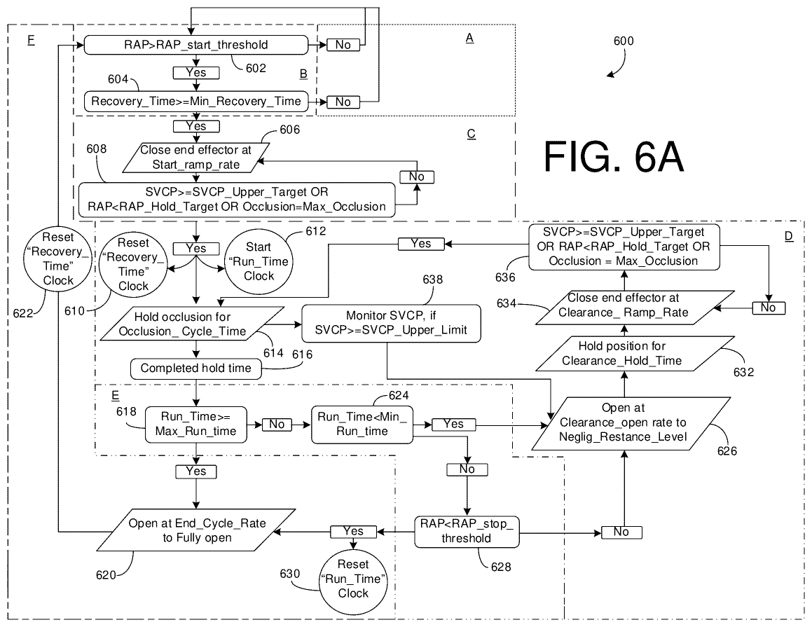

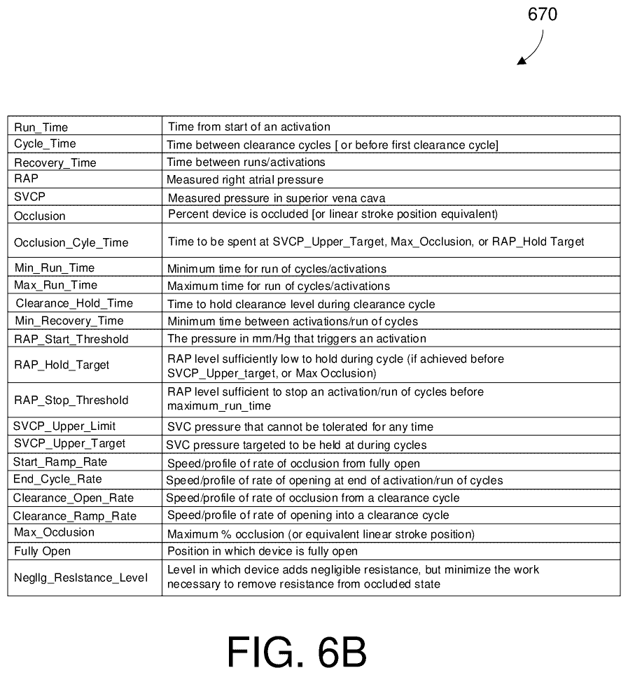

In some examples, a range of collapsing or expanding is selected based on one or more of: a predefined occlusion profile, a predefined occlusion schedule, a differential between the first pressure and the second pressure, and a detected rate of increase in the second pressure. The predefined occlusion profile may include one or more parameters (see FIG. 6B) and/or rules for an occlusion cycle. The predefined occlusion schedule may represent an occlusion cycle time and/or per day or per hour based actuation algorithm for the device 100. The detected differential between the first pressure and the second pressure may be used to trigger more or fewer collapsing or expanding events for the membrane 112. The detected rate of increase in the second pressure may be detected by the sensor 142, for example, and used to trigger collapsing or expanding events for the membrane 112.

An example rate of increase in the first pressure (e.g., RAP) that may cause the occlusion device to begin occluding at a rate of about 5% occlusion per minute, or as fast as fully occluded in less than about 5 seconds.

An example rate of increase in the first pressure (e.g., RAP) that may cause the occlusion device to stop occluding may be at a rate of about −5% occlusion per minute to about fully open in less than about 5 seconds.

An example rate of increase in the second pressure (e.g., SVC upstream from the detected RAP) that may cause the occlusion device to begin occluding at about a rate of 25% occlusion per minute to about fully occluded in less than about 5 seconds; or a rate of about −5% occlusion per minute to fully open in less than about 5 seconds.

An example rate of increase in the second pressure (e.g., SVC upstream from the detected RAP) that may cause the occlusion device to stop occluding at a rate of about 5% occlusion per minute to fully open in less than about 5 seconds.

In some examples, actuating the occlusion device 100 (e.g., actuating the membrane 112) may be performed in response to monitoring pressure. For example, device 100 may be programmed to monitor pressure using one or more processors, sensors, and/or instructions. In some examples, the membrane 112 may be triggered to radially collapse at the outflow end and toward the central axis (C) of the expandable frame 106 in response to detecting the first pressure is above a predefined pressure threshold. In some examples, the membrane 112 may be triggered to radially expand (at or near at least a portion of the membrane) away from the central axis (C) of the expandable frame 106 in response to determining that the second pressure is increasing at or above a predefined rate. In this way, the device 100 may use the second pressure as a feedback loop to cause actuation of the membrane 112 to modulate the first pressure. In some examples, the blood vessel is a superior vena cava, the first pressure is RAP, and the second pressure is SVC pressure indicating a level of intracranial venous pressure of a subject implanted with the device 100.

In some examples, the monitoring of the first pressure and the monitoring of the second pressure may be performed substantially continuously. In such examples, the monitoring may further include generating an indication to adjust the membrane 112 to a selected one of a plurality of positions between expanded and collapsed in response to detecting, at a second time period, that the first pressure is at or below a predefined pressure threshold. In some examples, the plurality of positions between expanded and collapsed may include at least an expanded position configured to allow the blood flow through the blood vessel, a partially expanded position configured to partially occlude the blood vessel, and a collapsed position configured to block the outflow end to occlude the blood vessel.

One or more of the plurality of positions may be selected based at least in part on the first pressure or the second pressure detected during the first time period. The predefined pressure threshold may include a level or range of a pressure threshold, as described in detail elsewhere herein.

In some examples, monitoring of the first pressure and the second pressure may include detecting (by onboard processors, sensors, etc.) when the first pressure is above a first predefined pressure range, actuating the membrane 112 to modulate the blood flow through the blood vessel or an adjacent blood vessel according to a first actuation cycle for maintaining the first pressure within the first predefined pressure range (described elsewhere herein). For example, device 100 may be operated in a first actuation cycle to maintain the first pressure. During the cycle, the membrane 112 may be actuated to modulate blood flow through the SVC (or an adjacent vessel) to control RAP, for example. The monitoring may also include detecting when the second pressure is above a second predefined pressure threshold associated with pressure in the SVC, switching the device 100 (e.g., membrane 112) to a second actuation cycle to alter the modulation of the blood flow through the blood vessel, and reverting to the first actuation cycle when the second pressure is detected to be within the second predefined pressure range (described elsewhere herein). In this way, the device 100 may perform any number of actuation cycles based on detected pressures.

In some examples, the first actuation cycle is performed for a first time period (e.g., about 30 minutes to about 3 hours) and the second actuation cycle is performed for a second time period (e.g., about 15 minutes to about 30 minutes) to maintain the first pressure within the first predefined pressure range and the second pressure within the second predefined pressure range. In some examples, the first actuation cycle may be modified based on the first pressure in the blood vessel, the second pressure in the blood vessel, and one or more of a determined cardiac pulsatility measured by the device 100 when implanted into the blood vessel, a respiratory effect detected by the device 100 when implanted into the blood vessel, a physiological effect detected by the device 100 when implanted into the blood vessel, and an activity exertion level detected by the device 100 when implanted into the blood vessel. For example, the signal processing of pressures and/or time may be assessed and/or obtained from measuring pulsatility and respiratory effects. Exertion may be assessed and/or obtained using one or more additional sensors (e.g., accelerometer). In some examples, the physiological effects and/or activity exertion levels may be determined using pattern recognition assessment of variations in RAP (and/or SVCP) as a result of exercise, for example.

In some examples, actuating the membrane 112 may be performed based on a determined elapsed time in which the monitored first pressure is within a predefined pressure range. The elapsed time may be determined based at least in part on the monitored first pressure and the monitored second pressure over a specific monitoring time or over an occlusion cycle (or number of cycles).

In some examples, actuating the membrane 112 to at least partially occlude the blood vessel may include selecting an occlusion level for the device 100 according to the detected first pressure and occluding the blood vessel according to the selected occlusion level. Example occlusion levels may be represented as a percentage from about zero percent occluded to about 100 percent occluded. For example, an occlusion level may be selected to occlude a blood vessel by about 75% to about 80%, about 80% to about 85%, about 85% to about 90%, about 90% to about 95%, and/or about 95% to about 100%. The occlusion may be continuous at one of the above levels, for example, until a predefined right atrium pressure drop is detected or until a safety threshold has been reached at the SVCP side of the occluding device.

In some examples, the occlusion level may be increased in response to detecting that the first pressure is above a predefined pressure range for a time exceeding a predefined time threshold. For example, the predefined time threshold may represent a time associated with an occlusion cycle time, a time associated with executing multiple occlusion cycles, or an absolute time or time range. In some examples, the predefined pressure range for the first pressure may be about 2 mmHg to about 10 mmHg. In some examples, the predefined pressure range for the first pressure may be about 8 mmHg to about 10 mmHg. One skilled in the art will appreciate that other pressure ranges may also be utilized.

In some examples, the occlusion level is decreased in response to detecting that the second pressure is above a second predefined pressure range at a time after the predefined time threshold. The predefined time threshold may represent a time associated with an occlusion cycle time, a time associated with executing multiple occlusion cycles, or an absolute time or time range. In some examples, the second predefined pressure range for the second pressure may be about 10 mmHg to about 25 mmHg. In some examples, the second predefined pressure range for the second pressure may be about 15 mmHg to about 20 mmHg. One skilled in the art will appreciate that other pressure ranges may also be utilized.

In some examples, the occlusion level is selected to maintain a first predefined pressure range for the first pressure in the blood vessel and maintain a second predefined pressure range for the second pressure in the blood vessel for at least one of about 70 percent to about 75 percent; about 75 percent to about 80 percent; about 80 percent to about 85 percent; about 85 percent to about 90 percent; or about 90 percent to about 95 percent of a predefined cycle time associated with the monitoring. For example, rules and/or parameters described elsewhere herein may be used to ensure that a subject has pressures that are in range with healthy and/or predefined pressure levels for about 80 percent of the time indicated for monitoring the subject.

In some examples, monitoring of the first pressure and the second pressure may also include communicatively coupling the device 100 to a first external computing device (e.g., device 805a) and/or a second external computing device (e.g., device 805b), transmitting, to the second external computing device 805b, output data corresponding to the monitored first pressure and the monitored second pressure, and receiving, from the second external computing device 805b (based on the transmitted output data), health-based instructions. The health-based instructions may be triggered for display on the first external computing device 805a or another computing device. In some examples, the health-based instructions may include one or more of: instructions to titrate medication, instructions to visit a clinic, instructions to deliver rescue therapy, instructions to perform physical movements, instructions to modify the diet (e.g., water intake, potassium or salt intake), or instruction to perform stress reduction activities (e.g., mindful breathing, meditation, etc.).

In some examples, the output may include a time-in-range calculation, as described elsewhere herein. In such examples, the detecting of the first pressure and the detecting of the second pressure may be performed over a predefined time period (e.g., 2 minutes, 10 minutes, 1 hour, 24 hours, etc.). The time-in-range calculation may include determining an amount of time in which the first pressure is within the first predefined pressure range and the second pressure is within the second predefined pressure range divided by the predefined time period. Output data may be generated for the patient based on the time-in-range calculation. For example, the output data may include the time-in-range calculation determined for the predefined time period. Example output data based on the time-in-range calculation may include, for example, health-based instructions. The health-based instructions may be triggered for display on the first external computing device 805a or another computing device. In some examples, the health-based instructions may include one or more of: instructions to titrate medication, instructions to visit a clinic, instructions to deliver rescue therapy, instructions to perform physical movements, instructions to modify the diet (e.g., water intake, potassium or salt intake), or instruction to perform stress reduction activities (e.g., mindful breathing, meditation, etc.).

In some examples, monitoring of the first pressure and the second pressure may include monitoring a rate of increase in the first pressure over a first time period and monitoring a rate of increase in the second pressure over the first time period, and actuating the membrane 112 after the first time period based on the rate of increase in the first pressure or the rate of increase in the second pressure. In this example, the rate of increase in the second pressure may indicate that SVC pressure is increasing at a rate that may cause intracranial venous pressure to rise in a subject and put the subject at risk. The second pressure may be used to trigger the device 100 to stop occluding the SVC or reduce an occlusion amount of the SVC to alleviate the SVC pressure upstream of the occlusion site. Sensors 140 and 142 may be used to detect such pressures to ensure the device 100 is operating to help the subject rather than causing undue increased pressures in the venous system.

In some examples, actuating the membrane 112 after the first time period may include actuating the membrane 112 to occlude the blood vessel until the first pressure is determined to be within a first predefined pressure range and modifying an occlusion level of the membrane in response to determining that the second pressure exceeds a second predefined pressure range, as described in detail elsewhere herein. In such an example, the second predefined pressure range may be about 15 mmHg to about 20 mmHg and the first predefined pressure range may be about 8 mmHg to about 10 mmHg.

In some examples, the device 100 is an implantable system for alleviating pressure within a blood vessel. The system may include a device (e.g., with a membrane 112) for modulating a flow of blood through the blood vessel, a first sensor (e.g., sensor 140) electrically coupled to the device, a second sensor electrically coupled to the device, and a processing module electrically coupled to the first sensor 140 and the optional second sensor 142. The first sensor 140 may detect a first pressure in a blood vessel. The optional second sensor 142 may detect a second pressure in the blood vessel at a location upstream of the first sensor. The processing module may execute instructions including monitoring outputs from the first sensor and the second sensor and actuating the device to perform blood flow modulation through the blood vessel based on the monitoring of the outputs. The outputs may be pressure measurements obtained over time from sensors on device 100, for example.

FIG. 1B illustrates a side view of the example flow modulating device of FIG. 1A. In this example, the device 100 is shown with the membrane 112 in a partially collapsed state. The partially collapsed state may represent a restricted blood flow state in which the membrane 112 radially collapses toward the central axis (C) of the frame 106 to reduce (or stop) blood flow through the blood vessel. Such a state may allow for a partial flow of blood, for example, through a lumen associated with the membrane 112. FIG. 1A, by contrast depicts the device 100 in an unrestricted blood flow state in which the membrane 112 is depicted radially expanded away from the central axis (C) of the expandable frame 106 to allow blood to flow through the blood vessel in which device 100 is implanted.

Positioning the membrane 112 in the partially collapsed state (e.g., a restricted blood flow state) shown in FIG. 1B, the control wire 122 may be actuated by a control element 124 coupled to, or otherwise in communication with, the control wire 122 to cause tensioning of the control wire 122 and closure or partial closure (e.g., cinching) of the membrane 112 at the outflow end 116. For example, the membrane 112 may be adjustable to form a cinched portion at the outflow end 116. For example, the cinching to form a cinched portion may include causing a perimeter of the outflow end 116 to be pleated, folded, or otherwise collapsed toward the central axis (C) by tensioning the control wire 122, which may result in reversibly reducing or closing the cross section at the outflow end 116. Such cinching of the perimeter of the outflow end 116 may be performed by device 100 to fully collapse the outflow end 116 of the membrane 112 resulting in occlusion of the blood vessel associated with the device 100. The cinching of the perimeter of the outflow end 116 may also be performed by device 100 to partially collapse the outflow end 116 of the membrane 112 resulting in a partial occlusion of the blood vessel associated with the device 100.

In general, actuating the control wire 122 may result in positioning the membrane 112 and/or device 100 in an unrestricted blood flow state or a restricted blood flow state. The device 100 may include an actuation device (not shown), either active or passive, to actuate the control wire 122. The actuation device may use a power source associated with or coupled to device 100 to induce changes in blood flow states of the membrane 112 and/or other portion of device 100. In some examples, the actuation device may use passively induced movement. For example, passively moving a portion of the device 100 may include manually actuating pull wires (e.g., sutures, actuation wires/cords/elements, etc.) and/or anatomy responses (e.g., changes in vessel inner diameter, intra-vessel pressure, etc.

In some examples, the actuation device for actuating the control wire 122 of the device 100 may include an actuator coupled to the control wire 122 of the device 100, a first magnet to induce rotation of the actuator, and a control device communicatively coupled to the actuator. In some examples, the first magnet is a permanent magnet, and the second magnet is a permanent magnet. In some examples, the first magnet is a permanent magnet, and the second magnet is an electromagnet. In some examples, the actuation device may be a magnetically driven actuator. In such an example, the control device may include a second magnet for generating a changing magnetic field pole direction to cause rotation of the first magnet and operation of the control wire 122 and device movement to the unrestricted blood flow state or to the restricted blood flow state. For example, the actuation device may cause rotation of the second magnet in a first direction to induce rotation of the first magnet, thereby causing the actuator to tension the control wire 122 to cause the membrane 112 to radially collapse inward and toward the central axis (C) at the outflow end 116. For example, the first direction of rotation of the second magnet may attract the first magnet.

The actuation device may also cause rotation of the second magnet in a second direction to induce rotation of the first magnet, thereby causing the actuator to release tension in the control wire 122 to cause the membrane 112 to radially open at the outflow end 116. For example, the second direction of rotation of the second magnet may repel the first magnet.

In some examples, the control device is implanted in the same subject in which the device 100 is implanted. The control device may be implanted adjacent to the device 100 or remote from the device 100. In some examples, the control device is implanted subcutaneously in the subject. In some examples, the control device is disposed external to a body of the subject.

In operation, the device 100 may receive a signal from an actuator that triggers the control element 124 to cause actuation of the control wire 122 and in turn causes a radial collapse of the membrane 112 at the outflow end 116. Such an actuation of the control wire 122 may cause the control wire 122 to be tensioned and to pull the eyelets radially toward the central axis (C) to collapse or partially collapse the membrane 112. In addition, the outflow end 116 of the membrane 112 is configured to radially expand away from the central axis (C) of the expandable frame 106, in response to an actuation of the control wire 122. The control wire 122 may be actuated by the control element 124 connected to the control wire 122 in a similar fashion as described above to cause the control wire 122 to release the tension and to release the eyelets radially away from the central axis (C) to expand or partially expand the membrane 112. The signal received from the actuator may be triggered based on monitoring performed by processors and sensors of device 100.

While membrane 112, members 120a-120f, and eyelets 126a-126f are described as a cinching occlusion mechanism for the device 100, any occlusion mechanism, occlusion element, and/or end effector may be employed with the methods described herein. In general, one or more control elements can be coupled to a body of an occlusion device. The control element and/or end effector to be moved (or actuated), can include one or more leaflets, flaps, valves, or valve portions (e.g., used to restrict blood flow through a blood vessel).

In some examples, the plurality of eyelets 126a-126f (or other cinching elements) may be utilized with the membrane 112 and without the elongate support members 120a-120f. For example, membrane 112 may be closed or opened by respectively cinching or releasing a wire threaded through eyelets 126a-126f. The membrane 112 in this example may be secured or otherwise anchored to a portion of the vessel without the structure provided by elongate support members 120a-120f.

The device 100 may include a power source (not shown) coupled to the control wire 122 or indirectly coupled to the control wire. The power source may include a battery or a wall outlet that may be electrically connected to the control wire 122 or another portion of device 100. The electrical connection may allow active powering of device 100 operations. In such an example, a processor may be utilized to send and/or receive signals to activate device operations via the actuation device. In some examples, the actuation device can be configured to send a first signal to the control wire 122 to activate application of tension to the control wire 122. For example, a processor (not shown) may be programmed to trigger tensioning of the control wire 122 in response to detecting a particular condition of the blood vessel or the device 100. The tensioning of the control wire 122 may result in cinching the outflow end 116 of the membrane to place the device in a restrictive blood flow state. Similarly, the actuation device can be configured to send a second signal to the control wire to activate releasing of the tension from the control wire 122 in response to detecting another condition of the blood vessel or the device 100. For example, a processor (not shown) may be programmed to trigger a release of tension in the wire 122 in response to detecting a particular condition of the blood vessel or the device 100. The release of the tension of the control wire 122 may result in uncinching the outflow end 116 of the membrane to place the device in an unrestrictive blood flow state.

In some examples, when a flow modulating device is in a partially or substantially fully closed, occluded, or restricted state (e.g., one or more membranes 112 are partially or fully expanded at the outflow end 104), blood may pool, exhibit stasis, or create eddies at or proximal to an upstream or inflow end 102 and/or at a downstream or outflow end 104 of the flow modulating device (e.g., flow modulating device 100). This pooling, stopping, or slowing of blood flow may create one or more stasis zones within the IVC, SVC, or peripheral vessel. For example, these stasis zones may be created where the membrane 112 couples to the frame 106; where the membrane 112 and frame 106 together define a pocket, groove, indentation, or concave section; where the membrane 112 contacts a support member 120a-120f, and the like. To alleviate blood stasis within a potential stasis zone, a flow modulating device may include one or more stasis reduction solutions. For example, a flow modulating device 100 may include the membrane 112, as shown in FIG. 1C, that may include one or more potential stasis zones 130a, 130b positioned between a vessel wall 132a, 132b and an outside surface 134a, 134b of the membrane 112.

To alleviate blood stasis and/or pooling, stasis reducing (or stasis mitigating) features may be included on membrane 112 to ensure that blood may flow through the one or more of the potential stasis zones 130a, 130b. For example, FIG. 1D illustrates a side view of the example flow modulating device of FIG. 1A including one or more stasis reducing features. The stasis reducing features in this example include a plurality of grooves 138 that may function as conduits (e.g., gutters, paths, etc.) to move blood along a surface of the membrane 112 and/or the frame 106. The grooves 138 are shown on the membrane 112 in a spiral shape along the outer surface of a portion of the membrane. One skilled in the art will appreciate that more or fewer grooves may be provided on membrane 112 and such grooves may be angled at any angle to promote blood flow from a particular stasis zone. That is, the grooves 138 may be configured at an angle to enable blood to flow along portions of the expandable frame 106 and/or membrane 112 from the inflow end 102 to the outflow end 104 to reduce blood stasis around the implantable device having the grooves 138 therein. The grooves 138 may define a stasis reducing flow path that extends substantially spirally or substantially helically around central axis (C) and along at least a portion of the outer surface of the membrane 112. One skilled in the art will appreciate that other stasis mitigating features are possible.

In some examples, the blood stasis (e.g., pooling, stopping, or slowing of blood flow) may be detected by one or more of the sensors described herein. In response to detecting blood statis, the implanted device 100 may perform one or more agitation cycles to deter the implanted device 100 from remaining stagnant and accumulating thrombus. In some examples, the implanted device 100 may perform one or more agitation cycles to deter the implanted device 100 from adhering to surfaces in which the device 100 encroaches or moves upon during operation. The agitation cycle may be performed when the device 100 is in an occluded state, a partially occluded state, and an open state. The agitation cycle may include operation of one or more components of device 100. For example, any two or more of support members 120a-120f and control wire 122 may be operated (e.g., actuated) to induce an agitation cycle. In some examples, an agitation cycle may include electrical, chemical, or physical stimulation of any portion of the device 100.

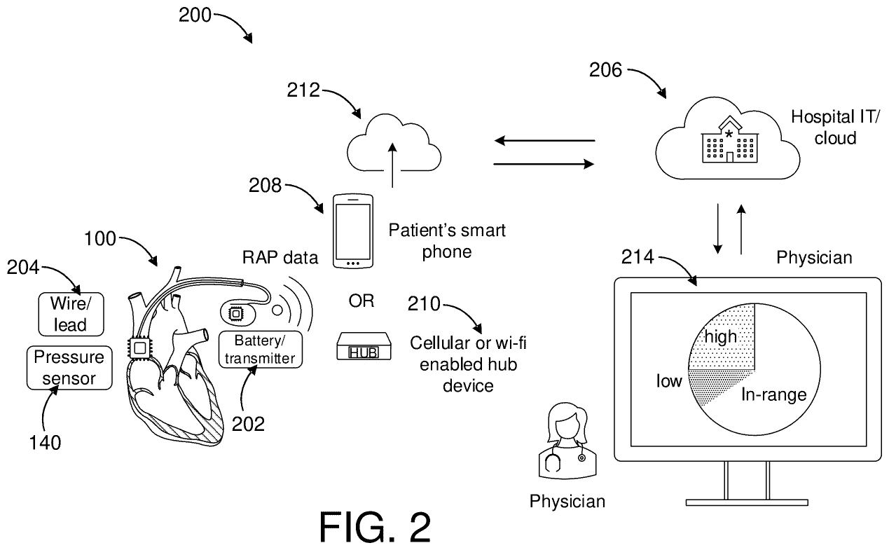

FIG. 2 is an example system 200 for monitoring and reducing pressures in a blood vessel. In this example, an occlusion device, such as device 100 may be implanted in the SVC (or an adjacent vessel) and arranged to capture (e.g., detect, monitor, etc.) RAP pressure and modulate blood flow based on the RAP pressure. For example, device 100 may include at least one processor (not shown), at least one pressure sensor (e.g., sensor 140) and optionally a second pressure sensor (e.g., sensor 142). The device 100 may also include a communication source (e.g., coil, antenna, etc.), a power source (e.g., battery/transmitter/controller circuit 202), and a control wire 204. The system 200 may further include one or more external devices. For example, system 200 may include a first external device (e.g., cloud server 206) and optionally a second external device (e.g., smartphone 208 and/or hub device 210). The system 200 may be in wireless communication with smartphone 208, hub device 210, cloud platform 212, and/or cloud server 206 to share pressure measurements, device configurations, operational parameter changes, user interface content 214, or other data shareable between the devices of system 200. The system 200 may include any number of processors amongst the devices of system 200.

Data shared from device 100 may be used to calculate time-in-range RAP metrics for a patient in which device 100 is implanted. The calculations may be performed on a continuous basis, a near continuous basis, or an on demand basis based on a system in range threshold programmed at the time of device implant or at another time after device implant, for example, according to patient specific attributes. An example system in range threshold may be about 2 mmHg to about 10 mmHg; about 2 mmHg to about 8 mmHg; about 4 mmHg to about 7 mmHg; about 8 mmHg; less than about 8 mmHg, etc. A processor onboard device 100 may obtain raw RAP data from sensors 140, 142 representing data captured from a pressure sensor placed in the right atrium (RA) or in proximity of the RA (e.g., the SVC). In a non-limiting example, if the subject spent 12 of the most recent 24 hours with an RAP of less than about 8 mmHg, then a determined time-in-range calculation for the subject would be about 50%.

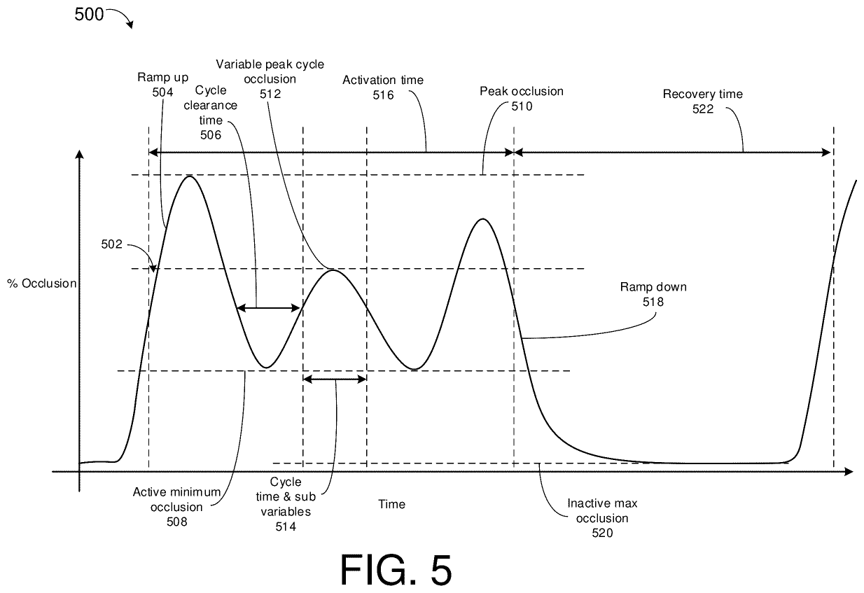

In operation, system 200 may capture RAP data and store such data on device 100. The data may be wirelessly transmitted to the smartphone 208 and/or the hub 210. This data transmission can occur periodically (e.g., intermittently, on a schedule, according to a fixed cadence, etc.) and/or when the implanted sensor 140 (and/or optionally sensor 142) is within a predefined physical range of the smartphone 208 or hub 210. The data may then be uploaded to the cloud platform 212 through a wired connection or wirelessly, for example via Wi-Fi or cellular connectivity. The RAP data may be transferred from the cloud platform 212 to the cloud server 206 (e.g., a hospital or clinic cloud or IT infrastructure). Alternatively, the RAP data may be sent directly to the cloud server 206 and/or through another third-party cloud platform, depending on available access for the device 100. Data encryption and decryption steps may occur along the process. The RAP data may be downloaded to a physician user interface (UI) showing user interface content 214, for example, for remote monitoring purposes. This real time (or near real time) assessment of congestion risk can enable a physician to take timely medical action including, but not limited to, titrating medications remotely, advising the subject to come into the clinic, delivering rescue therapy (e.g., home-based IV diuresis), etc. With the near continuous temporal resolution of RAP data, the systems described herein may identify excursions, inflection points, or trends that may correspond with positive or negative lifestyle or medication adherence behaviors of the subject. Such insights could be used to make HF self-management coaching more effective by tying specific behaviors to specific hemodynamic patterns. In some examples, a physician may interpret such RAP data and provide insights to provide health-based suggestions and diagnoses. Multiple different time-in-range RAP thresholds may be used to indicate various cardiovascular health levels/zones, to provide visibility on subject status and inform clinical decision making. Other parameters (as shown in FIGS. 5-7B) may also be tuned to modify operation of the device 100 and system 200.

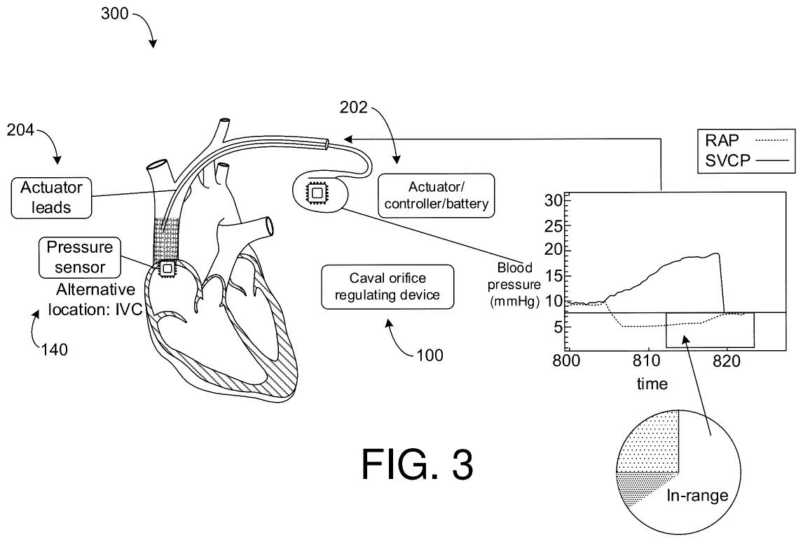

FIG. 3 is an example system 300 for managing time-in-range right atrial pressure for a subject. In this example, the time-in-range RAP metric can be used as a closed-loop feedback signal to the device 100 implanted in the vena cava (e.g., in the SVC or the IVC) which can dynamically occlude the vessel. The raw RAP data may serve as a basis of the metric calculation may be sourced from the pressure sensor 140. The sensor 140 may be either integrated with the body of the dynamic occlusion device 100 or as a separate component. In either scenario, the pressure sensor 140 may be positioned in the proximity of the RA junction (either SVC or IVC) to collect an accurate RAP measurement. The controller circuit 202 can modulate the algorithm of the duty cycle, occlusion ramp up/down speeds, and occlusion level of the occlusion device 100 to optimize for a maximum time-in-range RAP. This algorithm optimization can be a dynamic process, as described elsewhere herein. The algorithm optimization may be periodically updated as the algorithm adapts to the pressure patterns and responses from various occlusion permutations for the subject. Artificial intelligence and/or machine learning algorithms may be used to enable this adaptive learning using labeled datasets of occlusion parameters versus pressure patterns and/or pressure response patterns.

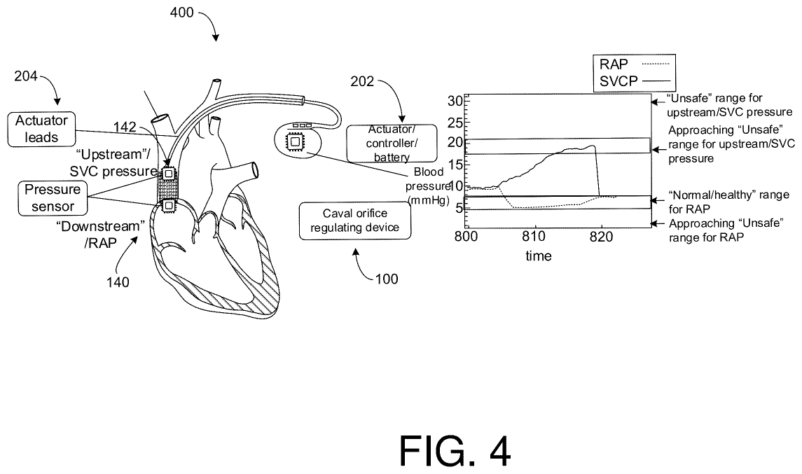

FIG. 4 is an example system 400 for maximizing time-in-range right atrial pressure while minimizing a time in which the SVC exhibits increased pressure. In this example, device 100 may be implanted in the SVC and be positioned to perform intermittent venous occlusion as a therapy, for example. Time-in-range RAP measurements can be captured (e.g., detected by sensor 140) in conjunction with SVC pressure measurements (e.g., detected by sensor 142). The raw RAP data may serve as a basis of the metric calculation. Other parameters (as shown in FIGS. 5-7B) may also be tuned to modify operation of the device 100 and system 300.

In this example, the sensor 140 for detecting RAP/downstream pressure sensor may be positioned in proximity to an SVC-right atrium (i.e., SVC-RA) junction in order to collect an accurate RAP measurement. The sensor 142 for detecting the SVC pressure sensor may be positioned upstream of the device 100 (and upstream of sensor 140). The controller circuit 202 can modulate the algorithm of the duty cycle, occlusion ramp up/ramp down speeds, and occlusion level of the occlusion device 100 in order to optimize for the balance between a maximum time-in-range RAP threshold and a minimum time in an unsafe SVC/upstream pressure threshold. In some examples, one or both thresholds may be programmed by a physician overseeing measurements from the device 100. Multiple different time-in-range RAP thresholds and SVC/upstream pressure thresholds may be used to indicate various cardiovascular health levels/zones, to provide visibility on subject status and inform clinical decision making. Other parameters (as shown in FIGS. 5-7B) may also be tuned to modify operation of the device 100 and system 400.

This algorithm optimization can be a dynamic process that may be periodically updated as the algorithm adapts to the pressure patterns and/or pressure responses from various occlusion permutations. Artificial intelligence and/or machine learning algorithms may be used to enable this adaptive learning using labeled datasets of occlusion parameters versus pressure patterns and/or pressure response patterns. The time spent in elevated upstream/SVC pressure range can also be transmitted to the physician and/or subject user interface, in order to provide visibility into any cerebral safety risks.

In any of systems 200, 300, 400, 800, 900, and 1050, the RAP time in range may be a true mathematical time in range, and/or it may be an effective time in range that may include steps such as performing noise filtering and/or removal of spurious data, or adjustment of the target range according to one or more of a determined cardiac pulsatility measured by the implantable device, a respiratory effect detected by the implantable device, a physiological effect detected by the implantable device, and/or an activity exertion level detected by the implantable device. For example, cardiac pulsatility may or may not be considered in the RAP assessments described herein. Respiratory effects may include variations in respiration that may or may not be considered in the RAP assessments described herein. Physiologic effects (e.g., coughing, hiccups, sneezes, etc.) include variations that may or may not be considered in the RAP assessments described herein. Conditions of higher than normal activity (e.g., exertion) may include activities that can cause filling pressures to rise in subjects, and the time in range may identify and/or consider a higher range as the target range during these periods when assessing the RAP of the subject using the devices described herein.

In some examples, non-physiological measurements may be captured by the sensors. Such measurements may or may not be considered in the RAP assessments described herein. For example, the devices described herein may identify and redact detected non-physiological variations from the RAP data to avoid making occlusion/pressure changes to the device when particular non-physiological variations are detected (e.g., exercise, atypical physical movements, or the like).

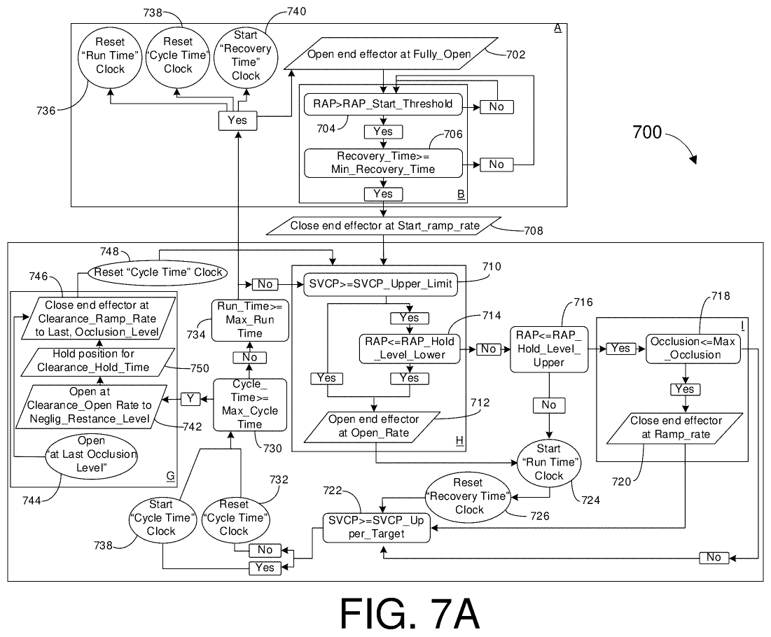

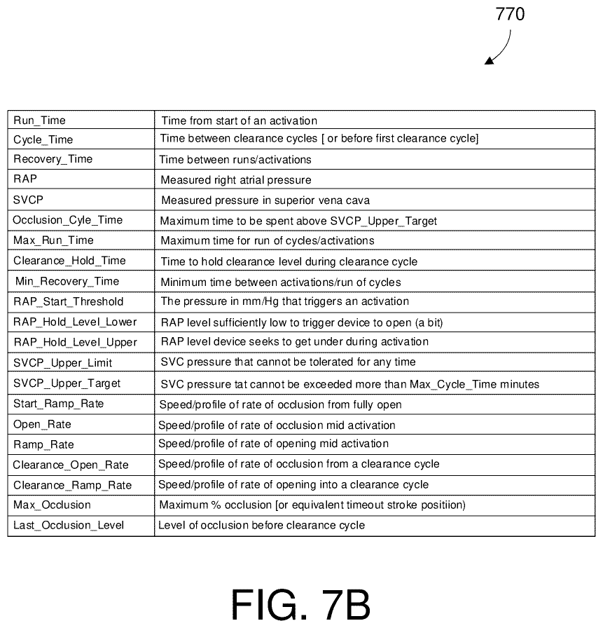

FIG. 5 is an example graph depicting an example cycle 500 of blood flow modulation using an implanted flow modulating device. The cycle 500 may be performed using any of the flow restricting devices described herein. As shown, the cycle 500 includes a signal 502 captured by a flow restricting device during operation of the device. The signal 502 is depicted as a percent of occlusion (y-axis) of the device over time (x-axis). A number of parameters (e.g., variables) may be used and/or modified to ensure that a particular occlusion device performs occlusion and de-occlusion of a blood vessel in a programmed or algorithmic fashion. For example, the parameters described below and/or depicted in FIGS. 5-7B may be used to configure actuation cycles for the flow modulating devices described herein. In general, any combination of the following parameters and/or the parameters in FIG. 5, table 650 (FIG. 6B), and table 770 (FIG. 7B) may be used to generate an operational occlusion cycle for the devices described herein.

The parameters may include a ramp up rate parameter 504 for modulating the flow of blood through the blood vessel. In some examples, the ramp up rate parameter corresponds to an amount of time to reach a selected percentage of occlusion by the device. For example, if an occlusion cycle is programmed to target an 80% occlusion of a blood vessel based on monitoring of the blood vessel performed by an occlusion device, then the ramp up time may be programmed to increase occlusion at a particular rate. That ramp up rate parameter 504 may be programmed to occur in a few seconds or up to a few minutes. In some examples, the ramp up rate parameter 504 may be programmed to occur at a time greater than about three minutes. In some examples, the ramp up rate parameter 504 may be used to program a state transition time for the occlusion device to ensure that the device transitions between occlusion states (or ranges) over a specified and predefined period of time. The ramp up rate parameter 504 may be set and reset according to monitoring performed by one or more sensors of any of the devices described herein. In some examples, the ramp up rate parameter 504 may be preset according to detected pressures, subject-specific anomalies or disease state, and/or physician instructions.

The parameters may include a clearance time parameter 506 corresponding to an amount of time for clearing blood volume from one or more portions of the device. For example, the device be programmed to open to allow clearance of a built up volume of blood and/or to minimize a time that blood is relatively static upstream from the device. In some examples, the clearance time parameter 506 may represent a cycle clearance time that represents a time in which the device is not occluding such that the blood vessel may return to flowing without intervention. Such a break in occlusion may allow for a reduction in pressure upstream of the occlusion site. Reducing upstream pressure may function to reduce cerebral pressure for a period of time (i.e., a clearance time indicated in the clearance time parameter). The clearance time parameter 506 may be set and reset according to monitoring performed by one or more sensors of one of the devices described herein. In some examples, the clearance time parameter 506 may be preset according to detected pressures, subject-specific anomalies or disease state, and/or physician instructions.

The parameters may include an active minimum occlusion parameter 508 that represents a period of time when the device is not in an occlusion (e.g., therapy) cycle. In some examples, the active minimum occlusion parameter 508 may be set to conserve or reduce battery usage of the device. In some examples, the active minimum occlusion parameter 508 may be used to arrange a baseline state of occlusion percentage. The baseline occlusion percentage may be programmed for a specific subject based on preliminary testing of the subject, monitoring of the subject, and/or other variable associated with the subject.

Example baseline occlusion levels may be about 60% to about 65%, about 65% to about 70%, about 70% to about 75%, about 75% to about 80%, about 80% to about 85%, about 85% to about 90%, about 90% to about 95%, and/or about 95% to about 100%. The active minimum occlusion parameter 508 may be set and reset according to monitoring performed by one or more sensors of one of the devices described herein. In some examples, active minimum occlusion parameter 508 may be preset according to detected pressures, subject-specific anomalies or disease state, and/or physician instructions.

The parameters may include a peak occlusion parameter 510 corresponding to a maximum occlusion capacity associated with operating the device. The peak occlusion parameter 510 may be set for the device to limit a maximum level of occlusion which may be defined by one or more of: maximum upstream pressure, minimum downstream pressure, and a predetermined maximum percentage of occlusion determined using a cross-sectional area of the vessel or assessing blood flow allowed through the device. This level may be conditional, meaning it could be a maximum for a particular state in which the program is operating in, or it may be an overall maximum level of occlusion enabled for the device. In some examples, the peak occlusion parameter 510 may be an absolute level of occlusion (e.g., about 50%, about 90%, etc.). The peak occlusion parameter 510 may alternatively be an individualized level based on the subject in which the device is implanted. For example, parameter 510 may be set or reset according to a predefined calibration based on one or more detected pressures for the subject. The peak occlusion parameter 510 may be set and reset according to monitoring performed by one or more sensors of any of the devices described herein. In some examples, peak occlusion parameter 510 may be preset according to detected pressures, safety measures, subject-specific anomalies or disease state, and/or physician instructions.

The parameters may include a variable peak cycle occlusion parameter 512 representing a way to vary the peak occlusion parameter 510 for one or more cycles of occlusion, for example. In this way, a different peak occlusion parameter 510 may be set for any number of selected cycles while retaining a setting for the peak occlusion parameter 510 in the remaining cycles. The variable peak cycle occlusion parameter 512 may be set and reset according to monitoring performed by one or more sensors of any of the devices described herein. In some examples, the variable peak cycle occlusion parameter 512 may be preset according to detected pressures, subject-specific anomalies or disease state, and/or physician instructions.

The parameters may include a cycle time parameter 514 corresponding to an amount of time for completing an occlusion cycle. For example, the cycle time parameter 514 may include a duty cycle (e.g., time interval) for how frequently the device is performing occlusion on the blood vessel. The cycle time parameter 514 may be set and reset according to monitoring performed by one or more sensors of any of the devices described herein. In some examples, the cycle time parameter 514 may be preset according to detected pressures, subject-specific anomalies or disease state, and/or physician instructions.

The parameters may include an activation time parameter 516 corresponding to an amount of time in which the device is actively operating (e.g., occluding or holding occlusion). The parameter 516 may refer to a run time representing an elapsed time of occlusion or an intervention period. The activation time parameter 516 may be used to manage an amount of time the device is active for the sake of energy conservation, minimizing an ability of the body of a subject to adapt and compensate for intermittent occlusions performed by the implanted device, and/or minimizing blood stasis around the device or thrombosis events around the device. The activation time parameter 516 may be set and reset according to monitoring performed by one or more sensors of any of the devices described herein. In some examples, the activation time parameter 516 may be preset according to detected pressures, subject-specific anomalies or disease state, and/or physician instructions.

The parameters may further include a ramp down parameter 518 representing a rate at which the device may be opening to begin to release an occlusion process (i.e., stop occluding the blood vessel). In some examples, the ramp down parameter 518 may be a rate that is measured as a decrease in percent occlusion per second. The rate may be characterized as a linear rate of decrease, or alternatively, a rate having a predefined rate profile. In some examples, the ramp down parameter 518 may be a rate of increase in right atrial pressure on a per second basis (e.g., mmHg/sec). In some examples, the ramp down parameter 518 may be a rate of decrease in an upstream pressure (e.g. an SVC pressure in mmHg). In some examples, the ramp down parameter 518 may be defined for different conditions, for example, a predefined rate/profile of ramp up for a decrease in occlusion mid activation (e.g., for a clearance cycle, or a change in conditions during the activation of device occlusion). In some examples, the ramp down parameter 518 may be defined as a final rate of opening (de-occlusion) as the device returns to an unrestricted or non-occluded state. The ramp down parameter 518 may be set and reset according to monitoring performed by one or more sensors of one of the devices described herein. In some examples, the ramp down parameter 518 may be preset according to detected pressures, subject-specific anomalies or disease state, and/or physician instructions.

The parameters may also include an inactive maximum occlusion parameter 520 representing a length of time in which the device is not in an occlusion (e.g., therapy) cycle. In this example, the blood vessel may be a patent (i.e., open) vessel. The inactive maximum occlusion parameter 520 may be set and reset according to monitoring performed by one or more sensors of one of the devices described herein. In some examples, the inactive maximum occlusion parameter 520 may be preset according to detected pressures, subject-specific anomalies or disease state, and/or physician instructions.

The parameters may further include a recovery time parameter 522 corresponding to a minimum time between actuations (i.e., occlusion cycles) performed by the device. For example, the recovery time parameter 522 may represent a time in between adjacent intervention/occlusion cycles such as a refractory period. The refractory period may be set for a time to allow the body to rest between occlusion cycles. For example, the time may range from about 30 minutes to about 3 hours; about 1 hour to about 2 hours; or about 3 hours to about 1 day.

In some examples, the recovery time parameter 522 may be selected to limit the amount of time the device is active for the sake of energy conservation, minimizing an ability of the body of a subject to adapt and compensate for the intermittent occlusions performed by the device, and/or minimizing blood stasis around the device or thrombosis events around the device. In some examples, the device is in an open (e.g., un-occluded or minimally occluded) state during this recovery time period. The recovery time parameter 522 may be set and reset according to monitoring performed by one or more sensors of any of the devices described herein. In some examples, the recovery time parameter 522 may be preset according to detected pressures, subject-specific anomalies or disease state, and/or physician instructions.

The parameters may further include an agitation cycle parameter (not shown) corresponding to an amount of time to allow the device to remain stationary before triggering agitation of one or more components of the device. The agitation cycle parameter may be a safety parameter to prevent thrombus and/or reduce blood stasis by ensuring the device is agitated or moved at a predefined interval. In some examples, the device may also operate an agitation cycle according to one or more parameters to keep the implanted device from remaining static. This agitation cycle may be performed when the device is occluded or open. In some examples, the agitation cycle may be performed passively to ensure the end effector of the occlusion device is continuously moving.

The parameters may further include a level of negligible resistance parameter (not shown) corresponding to a level of occlusion determined to have negligible impact on resistance to blood flow through the device. The parameters may further include and a hold time parameter (not shown) corresponding to an amount of time the occlusion is held at a particular occlusion level by the device.

FIG. 6A is a flow diagram depicting an example process 600 for assessing and modulating pressures in a blood vessel. The process 600 may function to monitor and reduce pressures associated with cardiac blood flow at a target region in a blood vessel of a heart of a subject. In some examples, the target region includes a portion of a vena cava of the subject, a portion of the superior vena cava of the subject, a portion of an inferior vena cava of the subject, or a portion of an adjacent vessel to the vena cava, the superior vena cava, or the inferior vena cava. In some examples, the process 600 functions to modulate a volume of blood flowing from the blood vessel into a right atrium to decrease right atrial pressure and/or SVC pressure associated with intracranial venous pressure of the subject. The process 600 may be used for blood flow regulation in the SVC or the IVC, but can additionally, or alternatively, be used for any suitable applications, clinical or otherwise. In general, process 600 may be used with any of the devices and/or systems described herein. For example, a processor 808 may carry out the steps of process 600.

The process 600 may include one or more processors performing monitoring and pressure assessment steps, according to particular timing zones. Example timing zones shown here include a Zone A, a Zone B, a Zone C, a Zone D, a Zone E, and a Zone F. Zone A represents a time in which the RAP is in range, thus the occlusion device is not operating and/or the device is in a recovery period. Zone B represents a time in which conditions are checked to begin actuating the occlusion device. Zone C represents a time in which the occlusion device is ramping up to an occlusion level. Zone D represents a time in which one or more occlusion cycles are actively occurring. Zone E represents a time in which conditions are checked to stop actuating the occlusion device and/or to hold the occlusion device. Zone F represents a time in which conditions are checked to cease actuating the occlusion device.

As shown in FIG. 6A, at step 602, the process 600 includes obtaining a RAP measurement and determining whether the RAP measurement is above a predefined start threshold (e.g., RAP_Start_Threshold parameter) to trigger activation of an occlusion cycle. If the RAP measurement is at or below the predefined start threshold, the process 600 may continually or intermittently continue to perform step 602. If the RAP measurement is above the predefined start threshold, then the process 600 triggers activation of an occlusion cycle.

During operation of the occlusion cycle, the process 600 may determine, at step 604, whether a time between occlusion cycles has elapsed beyond a minimum recovery time (e.g., Min_Recovery_Time parameter). If the time is not above or equal to the minimum recovery time, then the process 600 may continually, intermittently, or according to a schedule, continue to perform step 604. If the time is above or equal to the minimum recovery time, then the process 600 includes ramping up the occlusion in the occlusion cycle according to a ramp up rate (e.g., Start_ramp_rate parameter) representing a speed and/or profile of the rate of occlusion from fully open (i.e., non-occluding) device and move to Zone C, at step 606.

At step 608 and during step 606, the process 600 may determine whether (1) SVC pressure is above or equal to an upper target for the SVCP (e.g., SVCP_Upper_Target parameter) representing SVC pressure targeted to be held to during occlusion cycles (2) whether the RAP is less than a hold target (e.g., RAP_Hold_Target parameter) representing an RAP level sufficiently low to hold during an occlusion cycle (if achieved before an upper target SVCP limit or before Max occlusion), and/or (3) whether the occlusion is equal to the maximum occlusion (representing a maximum percentage of occlusion or equivalent linear stroke position). If none of (1), (2), and (3) are true, then process 600 may continually, intermittently, or according to a schedule, perform step 606 until one of (1), (2), and (3) becomes true.

If any of (1), (2), or (3) are true, then the process 600 may move to Zone D and may trigger either or both of a reset (at step 610) of a recovery clock (Recovery_Clock parameter) to begin elapsing time on a recovery clock and a run time clock (Run_Time parameter) (at step 612) to begin elapsing time on a run time clock during the occlusion cycle started in step 604.

At step 614, the process 600 may determine if a hold occlusion time has elapsed to the occlusion cycle time (e.g., Occlusion_Cycle_Time parameter) representing a time to be spent at SVCP_Upper_Target parameter, Max_Occlusion parameter, or RAP_Hold_Target parameter, as shown in table 670 of FIG. 6B. If the hold occlusion time has elapsed, then the hold time is completed at step 616 and the process 600 may move to Zone E.

At step 618, the process 600 may determine if the run time of an occlusion cycle is greater than or equal to a maximum run time parameter (e.g., Max_Run_time parameter). If the run time is greater than or equal to the maximum run time parameter, then the process 600 moves to Zone F to open the occlusion device according to an end cycle rate (e.g., End_Cycle_Rate parameter) representing a speed and/or profile of a rate at which to open the occlusion device at the end of activation and/or occlusion cycles. After indicating to open the device, the process 600 may start the recovery clock for the device, at step 622. The process 600 may then return to Zone B to detect whether or not the RAP is greater than a RAP start threshold.

If the run time is less than the maximum run time parameter, then the process 600 determines whether the run time parameter is less than the minimum run time (e.g., Min_Run_time parameter) at step 624. If the run time parameter is less than the minimum run time, the process 600 moves to Zone D and step 626 to determine whether a clearance open rate (e.g., Clearance_Open Rate parameter) has been met. If the rate has been met, the process 600 may open the occlusion device at the clearance open rate according to a negligible resistance level (e.g., Neglig_Resistance_Level parameter) representing a level in which the device adds negligible resistance, but minimizes the work used to remove resistance from the occluded state. If the run time parameter is less than the minimum run time, the process 600 determines, at step 628, whether the RAP is less than an RAP stop threshold (e.g., RAP_Stop_Threshold parameter) representing a RAP level sufficient to stop an activation of one or more occlusion cycles before reaching a maximum run time. If the RAP is greater than the RAP stop threshold, then the process 600 returns to the Zone F, resets the run time clock at step 630 and opens the occlusion device at step 620, which also starts the recovery time at step 622, and returns to step 602 to assess RAP.

Referring again to step 626, after opening the device at the clearance open rate, the process 600 may hold position for a clearance hold time (e.g., Clearance_Hold_Time parameter) representing a time to hold clearance level during a clearance cycle, at step 632 and close an end effector (e.g., membrane 112, a movable occlusion portion, an end effector, etc.), according to the clearance ramp rate (e.g., Clearance_Ramp_Rate parameter) representing a speed and/or profile of a rate of opening the device when beginning a clearance cycle.

At step 636, the process 600 may determine whether (1) SVC pressure is above or equal to an upper target for the SVCP (e.g., SVCP_Upper_Target parameter) (2) whether the RAP is less than a hold target (e.g., RAP_Hold_Target parameter) and/or (3) whether the occlusion is equal to the maximum occlusion. If none of (1), (2), and (3) are true, then process 600 may continually, intermittently, or according to a schedule, perform step 634 until one of (1), (2), and (3) becomes true. If any of (1), (2), or (3) are true, then the process 600 may move step 614 to hold occlusions.

Referring again to step 614, after holding occlusions for the occlusion cycle time, the process 600 may continually, intermittently, or according to a schedule perform step 638 to monitor SVC pressure until the SVC pressure is greater than or equal to the SVC pressure upper limit (e.g., SVCP_Upper_Limit parameter) representing a pressure in the subject that cannot be tolerated for any amount of time. When the SVC pressure reaches the upper limit, the process 600 moves to step 626 to open the occlusion at the occlusion site.

Step 606, step 614, step 620, step 626, step 632, and step 634 represent actions executed by the device 100 according to processing instructions from a processor (e.g., processor 808). Step 602, step 604, step 608, step 616, step 618, step 624, step 628, step 636, and step 638 represent monitoring steps carried out by sensors 140, 142, etc. onboard device 100 according to processing instructions stored on a processor (e.g., processor 808). The monitoring steps may assess measured inputs captured by sensors 140, 142, etc. as compared to configured parameters of the system, as described in detail herein. Step 610, step 612, step 622, and step 630 represent time/timer sets (e.g., starts) and/or resets that assess a measurement versus a configured parameter according to instructions stored on a processor (e.g., processor 808). FIG. 6B is an example table 670 representing a number of parameters that may be tuned to modify operation of the devices described herein.

FIG. 7A is a flow diagram depicting an example process for monitoring and modulating pressures in a blood vessel.

The process 700 may function to monitor and reduce pressures associated with cardiac blood flow at a target region in a blood vessel of a heart of a subject. In some examples, the target region includes a portion of a vena cava of the subject, a portion of the superior vena cava of the subject, a portion of an inferior vena cava of the subject, or a portion of an adjacent vessel to the vena cava, the superior vena cava, or the inferior vena cava. In some examples, the process 700 functions to modulate a volume of blood flowing from the blood vessel into a right atrium to decrease right atrial pressure and/or SVC pressure associated with intracranial venous pressure of the subject. The process 700 may be used for blood flow regulation in the SVC or the IVC, but can additionally, or alternatively, be used for any suitable applications, clinical or otherwise. In general, process 700 may be used with any of the devices and/or systems described herein. For example, one or more processors 808 may carry out the steps of process 700.

The process 700 may include one or more processors performing monitoring and pressure assessment steps according to particular timing zones. Example timing zones shown here include a Zone A, a Zone B, a Zone G, a Zone H, and a Zone I. Similar to process 600, Zone A represents a time in which the RAP is in range; thus, the occlusion device is not operating and/or the device is in a recovery period and Zone B represents a time in which conditions are checked to begin actuating the occlusion device. Zone G represents a time in which the device is executing a clearance cycle. Zone H represents a time in which the device is determining whether conditions to open the device (e.g., stop occluding) have been met. Zone I represents a time in which the device is determining whether conditions to close the device (e.g., begin or increase occluding) have been met.

Referring to FIG. 7A, the process 700 may begin by opening an end effector (e.g., membrane 112, a movable occlusion portion, etc.), at step 702. At step 702, the process 700 may include determining if a RAP (of a subject in which the occlusion device is implanted) is above a predefined RAP threshold (e.g., RAP_Start_Threshold parameter), representing a pressure (e.g., in mmHg) that triggers activation of the occlusion device. If the RAP is less than or equal to the RAP threshold, the process 700 may continue to monitor the RAP until the RAP is detected to be above the RAP threshold. If the process 700 detects that the RAP is greater than the RAP threshold, the process may determine, at step 706, whether a recovery time has elapsed. For example, the process 700 may determine whether the recovery time (e.g., Recovery_Time parameter) representing a time between activations of the occlusion device is greater than or equal to a minimum recovery time (e.g., Min_Recovery_Time parameter), representing a minimum time between activations and/or occlusion cycles. If the recovery time is not greater than or equal to the minimum recovery time, the process 700 may continue to perform step 704 and step 706 until both conditions are true. If the recovery time is greater than or equal to the minimum recovery time, the process 700 moves to step 708 to begin closing the end effector according to a start ramp up rate (Start_ramp_rate parameter), representing a speed and/or profile of a rate of occlusion from fully open end effector. For example, the device may begin and continue occluding at least a portion of the blood vessel.

During occlusion, the process 700 may assess, at step 710, whether SVC pressure is greater than or equal to an SVC pressure upper limit (SVCP_Upper_Limit), representing an SVC pressure that may not be tolerated by the subject for any amount of time. If the SVC pressure is at or above the upper limit, then the process 700, at step 712, may open the end effector according to an open rate parameter (e.g., Open_Rate parameter), representing a speed and/or profile of a rate of occluding the device midway through activation of device occlusion. If the SVC pressure is below the upper limit, then the process 700 may determine, at step 714 whether the RAP pressure is below or equal to a RAP hold lower level (e.g., RAP_Hold_Level_Lower parameter) representing an RAP level sufficiently low to trigger the end effector (e.g., of the occlusion device) to open. If the RAP pressure is below or equal to the RAP hold lower level, then process 700 may perform step 712 and open the end effector. If the RAP pressure is above the RAP hold lower level, then process 700 may determine, at step 716, whether the RAP is greater than or equal to a RAP hold upper level (e.g., RAP_Hold_Level_Upper parameter), representing an RAP level in which the device actively seeks to operate at (or beneath) during operation of the device (e.g., occlusion cycles). If the RAP is greater than or equal to the RAP hold upper level, then process 700 determines, at step 718, whether the amount of device occlusion is less than or equal to a maximum occlusion (e.g., Max_Occlusion parameter) representing a maximum percentage of occlusion allowed by the occlusion device. If the occlusion is determined to be above the maximum occlusion, then process 700 may close the end effector, at step 720, according to a ramp rate (e.g., Ramp_rate parameter), representing a speed and/or profile of a rate of opening the occlusion midway through activation of device occlusion. The process 700 may move to step 722 to determine whether the SVCP pressure is greater than or equal to an SVCP upper target (e.g., SVCP_Upper_Target parameter), representing an SVC pressure that cannot be exceeded more than a maximum number of cycle minutes (e.g., Max_Cycle_Time parameter).

Referring again to step 712, upon opening the end effector, the process may reset the run time clock at step 724 and reset the recovery time clock at 726 before moving to perform step 722.

Referring again to step 716, if the RAP pressure is not greater than or equal to the RAP hold upper level, then the process 700 may reset the run time clock for the occlusion cycle, at step 724 and reset the recovery time clock at step 726 before moving to step 722. Referring again to step 718, if the occlusion is not greater than the maximum occlusion, then the process 700 may perform step 722.

At step 722, if the process 700 determines that the SVCP is greater than or equal to the SVCP upper target, then the process 700 may start a cycle time clock, at step 728 and move to step 730. If the process 700 determines that the SVCP is less than the SVCP upper target, then the process 700 may reset a cycle time clock at step 732, and move to step 730.

At step 730, the process 700 may determine whether a cycle time is greater than or equal to a maximum cycle time (e.g., Max_Cycle_Time parameter) representing a maximum time to be spent above the SVC pressure upper target. If the process 700 determines that the cycle time is less than the maximum cycle time, then the process 700 may determine if the run time is greater than or equal to the maximum run time, at step 734. If the process 700 determines that the run time is greater than or equal to the maximum run time, then the process 700 may move to Zone A to reset the run time clock (at step 736), reset the cycle time clock (at step 738), and start the recovery time clock (at step 740) and begin monitoring the RAP and SVC pressures again after opening the end effector, at step 702. If the process 700 determines that the run time is less than the maximum run time, then the process may return to step 710 to monitor/determine whether or not the SVC pressure is above or equal to the SVC pressure upper limit.

Referring again to step 730, if the process 700 determines that the cycle time is less than the maximum cycle time, then the process 700 may open the end effector, at step 742, according to a predefined clearance open rate (e.g., Clearance_Open_Rate parameter) representing a speed and/or profile of a rate of occlusion from a clearance cycle using the predefined negligible resistance level. In some examples, the process 700 may instead open the end effector to a last configured occlusion level at step 744 and then close the end effector, at step 746, according to a clearance ramp rate (e.g., Clearance_Ramp_Rate parameter). The clearance ramp rate parameter represents a speed and/or profile of a rate of opening into a clearance cycle. The process 700 may further reset the cycle time clock, at step 748, before moving to step 710 to monitor/determine whether or not the SVC pressure is above or equal to the SVC pressure upper limit.

Referring again to step 742, after opening the end effector of the occluding device, the process 700 may hold a position of the occlusion according to a clearance hold time (e.g., Clearance_Hold_Time parameter), representing a time to hold a clearance level during a clearance cycle, at step 750. After such time, the process may close the end effector (at step 746), reset the cycle time clock (at step 748) before moving to step 710 to monitor/determine whether or not the SVC pressure is above or equal to the SVC pressure upper limit.

Step 702, step 708, step 712, step 720, step 746, step 742, and step 750 represent actions executed by the device 100 according to processing instructions from a processor (e.g., processor 808). Step 704, step 706, step 710, step 714, step 716, step 718, step 722, step 734, and step 730 represent monitoring steps carried out by sensors 140, 142, etc. onboard device 100 according to processing instructions stored on a processor (e.g., processor 808). The monitoring steps may assess measured inputs captured by sensors 140, 142, etc. as compared to configured parameters of the system, as described in detail herein. Step 724, step 726, step 728, step 732, step 744, step 748, step 736, step 738, and step 740 represent time/timer sets (e.g., starts) that assess a measurement versus a configured parameter according to instructions stored on a processor (e.g., processor 808). FIG. 7B is an example table 770 representing a number of parameters that may be tuned to modify operation of the devices described herein.

With respect to any of the parameters described in FIGS. 6A-7B, a range of values may be predefined for one or more parameters. The range may function to allow the devices described herein to operate according to patient-specific data, condition-specific data, or other specified data to customize operation of occlusion or de-occlusion of such devices. In some examples, enable zero or extreme values may be provided to configure any number of parameters as a way to nullify (e.g., effectively ignore) a parameter during operation of the device carrying out process 600, process 700, process 1100, and/or process 1200.

FIG. 8 is a block diagram of an example system 800 for modulating blood flow through one or more blood vessels. The system 800 may be used with any of the flow restricting devices described herein. As shown, the system 800 includes flow restriction controls 802 and at least one implantable device 804, each of which may be optionally communicatively coupled to a first external computing device 805a and/or a second external computing device 805a. The implantable device 804 may correspond to any of the flow restricting devices described herein.

The flow restriction controls 802 may include one or more sensors 806, one or more processors 808, one or more control devices 810, and one or more actuation devices 812. Optionally, the flow restriction controls may include a power source 814 that may be internal to the controls 802, internal to the implantable device 804, or external to both the flow restriction controls 802 and the implantable device 804. In some examples, the power source may be wired to flow restriction controls 802 or implantable device 804. In some examples, the power source may be remotely accessed (e.g., wirelessly) by flow restriction controls 802 or implantable device 804 via device 805a and/or device 805b.

The one or more sensors 806 may be optional. In some examples, a single sensor 806 is coupled to implantable device 804 (see FIG. 3). In some examples, a second sensor 806 is also coupled to implantable device 804 (see FIG. 1A, FIG. 4). In some examples, the sensors 806 represent microelectromechanical pressure sensors (e.g., MEMS). The one or more sensors 806 may function to sense (e.g., detect) properties of the blood in which the sensor(s) are disposed within. For example, the sensors 806 may detect blood pressure within the blood vessel and/or any other physiological or anatomical parameters or properties of the blood or vessel. The one or more sensors 806 may include one or more of an image sensor, a strain gauge, a piezoelectric sensor, a capacitance sensor, and/or a vacuum pressure sensor. In general, sensor signals from sensors 806 may be transmitted to control devices, device 805a, device 805b, and/or elements described herein via a wired or wireless connection. Additionally, and optionally, the sensors 806 may utilize one or more processors 808 to transmit data to remote computing devices. The transmitted data may include sensor measurements, device position data and/or statistics, actuation events, or any other data from the system.

In some examples, the sensors 806 may assess and/or recognize patterns of blood pressures for a patient over time. The patterns may be used as a trigger to perform one or more device occlusions of a blood vessel. In some examples, sensors 806 and processors 803 may perform pattern recognition that may statistically indicate a state of the patient (e.g., different grades of physical activity, illness, volume overload, arrhythmia, acute kidney injury, etc.) and such data (or patterns of data) may inform the activation of vessel occlusion or de-occlusion.

The processors 808 may include one or more microprocessors, microcontrollers, or the like, as described elsewhere herein. The control devices 810 may include active or passive controls including, but not limited to wires, sutures, operated switches, motor controllers, and/or antennas. In some examples, the control devices 810 may include external control devices including, but not limited to, remote computers, tablets, smart phones, and/or external control devices for powering and/or controlling the flow restriction controls 802.

The actuation devices 812 may include mechanically actuating devices, electrically actuated devices, electromechanically actuated devices, or a combination thereof. For example, actuation devices 812 may include any one or more of a wire, a suture, a pull wire, a linear actuator (e.g., a pneumatic linear actuator, an electromechanical linear actuator, or a hydraulic linear actuator), a magnet or coil, etc. The power sources 814 may include, but are not limited to, battery power, wall power, magnets, induction coils, or the like.

In operation of system 800, the actuation device 812 may be coupled to the control device 810, which may manipulate or move portions of the implantable device 804 based on one or more signals received from one or more sensors 806. In embodiments that utilize a processor 808, the processor 808 may be communicatively coupled to the one or more sensors 806, control devices 810, actuation devices 812, power source 814, and/or implantable device 804 to actuate the implantable device 804 into a restricted blood flow state, an unrestricted blood flow state, or any position therebetween.

Although restricted and unrestricted flow states/configurations or restricted and unrestricted device positions are described herein, it is within the scope of the present disclosure that any number of intermediate positions or states are contemplated and included herein, whether or not expressly indicated.

FIG. 9 is a schematic diagram of an example embodiment of a system 900 for modulating blood flow through a blood vessel. The system 900 may include a first magnet 906, an actuation device 908, and a control element 914. The first magnet 906 may be operatively coupled to the actuation device 908. The actuation device 908 may be operatively coupled to the control element 914 to effect movement of the control element 914. In some examples, the control element 914 is a membrane. In some examples, the control element 914 is a control wire. In some examples, the control element 914 is a catheter portion. In some examples, the control element 914 is a ring. In some examples, the control element may be coupled or include a processor 903 for controlling and/or monitoring the control element 914, for example.

The system may include a control device 902 operatively coupled to a second magnet 904. The control device 902 can include (or be coupled to) the processor 903, power source (a battery, a capacitor, wall outlet, or any other suitable power source), antenna, operated switches, and/or any other control devices. The control device 902 and second magnet 904 may be located externally, but proximal to a user. In some examples, the control device 902 and second magnet 904 may be implanted (e.g., subcutaneously, intravascularly, etc.). In some examples, both the first magnet 906 and the second magnet 904 may be permanent magnets. In some examples, the first magnet 906 is a permanent magnet and the second magnet 904 is an electromagnet.

The system 900 may include a first sensor 910. The first sensor 910 may sense one or more physiological or anatomical attributes at a first location within a blood vessel and output a signal to the control device 902 (and/or processor 903 directly). The control device 902 (and/or processor 903 directly) may output an activation signal to the actuation device 908 to tension or release tension in the control element 914.

Optionally, the system 900 may include a second sensor 912. The second sensor 912 may sense one or more physiological or anatomical attributes at a second location within the blood vessel (upstream from the first location) and output a signal to the control device 902 (and/or processor 903 directly). The control device 902 (and/or processor 903 directly) may output an activation signal to the actuation device 908 to tension or release tension in the control element 914.

The first sensor 910 and the optional second sensor 912 may provide outputs to processor 903 (and/or control device 902) based on one or more rules and/or parameters for operating the system 900 when implanted into a blood vessel. For example, the outputs of the sensor 910 and/or the outputs of the sensor 912 may trigger activation signals to be sent to the actuation device 908 according to the rules and parameters described elsewhere herein.

The second magnet 904, although external to the user or implanted at a second location (the implantable device being at a first location), may be placed operationally proximal to the first magnet 906. By doing so, the magnetic pole orientation of the second magnet 904 influences the magnetic pole direction of the first magnet 906. For example, a magnetic gear train may be generated between the second magnet 904 and the first magnet 906, such that when the control device 902 rotates the second magnet 904, the first magnet 906 is rotated in an opposing direction. Rotating the first magnet 906 induces movement in the actuation device 908, which tensions or releases tension in the control element 914 or moves the control element 914 to a restricted or unrestricted blood flow state, respectively.

The control device 902 may receive signals from sensor 910 and/or optional sensor 912. Such signals may be indicative of characteristics of blood flow in the blood vessel (e.g., blood pressure). For example, when the control device 902 receives a signal indicative of a measured pressure higher than a predefined level (or range), the control device 902 can cause the second magnet 904 to rotate based on direct or analyzed feedback from sensor 910 and/or optional sensor 912. The rotation of the second magnet 904 can cause the first magnet 906 to rotate, which may actuate the actuation device 908 to move the control element 914 towards a restricted blood flow state. Further, when the control device 902 receives a signal indicative of a measured pressure lower than the predefined level, the control device 902 may cause the second magnet 904 to rotate in an opposing direction based on direct or analyzed feedback from sensor 910 and/or optional sensor 912. The rotation of the second magnet 904 causes the first magnet 906 to rotate, thereby actuating the actuation device 908 to move the control element 914 into the unrestricted blood flow state.

In general, sensor signals from sensor 910 and/or optional sensor 912 may be transmitted to control devices or elements described herein via a wired or wireless connection. Additionally, and optionally, the sensor 910 and/or sensor 912 may utilize one or more processors (e.g., processor 903) to transmit data to remote (e.g., external) computing devices. The transmitted data may include sensor measurements, device position data and/or statistics, actuation events, or any other data from the system.

FIG. 10 is a schematic diagram of an example embodiment of a system 1050 for modulating blood flow through a blood vessel. Similar to FIG. 9, FIG. 10 shows a magnetic gear train for manipulation of a control element 914 of an implanted device. The control device 902 and second magnet 904, as in FIG. 9, may be located external to the user or implanted (e.g., subcutaneously, intravascularly, etc.). Similar to FIG. 9, the first magnet 906, actuation device 908, control element 914, and optional sensor 910 may be implanted within the user. Unlike FIG. 9, the embodiment of FIG. 10 includes an implanted (in some examples, implanted subcutaneously) repeater magnet 905. This repeater magnet 905 may be used to extend the operational distance between the second magnet 904 and the first magnet 906, as it is implanted at an appropriate position between the two. Additionally, the repeater magnet 905 may be used to increase the torsional force that can be applied by the magnetic gear train. Further contemplated embodiments may include a repeater module with a second power source operatively coupled to the repeater magnet 905 and capable of charging and/or powering the rotation of the repeater magnet 905. Further, the rotation direction of the second magnet 904 and first magnet 906 are now the same, not opposing one another as in the embodiment of FIG. 9. For example, when manipulating the control element 914 towards the restricted or unrestricted blood flow states, the second magnet 904 can be rotated in the same direction as the desired direction of the first magnet 906.

### Methods

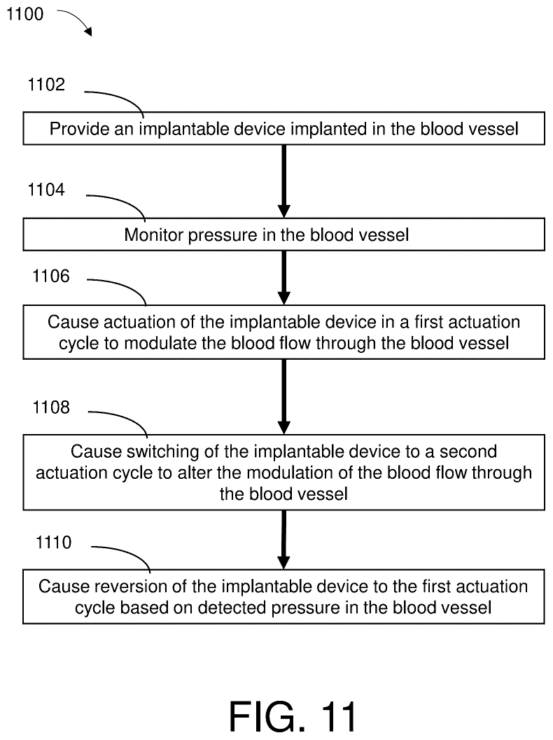

FIG. 11 is a flow diagram of an example process 1100 for modulating blood flow through one or more blood vessels. The process 1100 functions to monitor and reduce pressures associated with cardiac blood flow at a first target region in a blood vessel of a heart of a subject and at a second target region upstream to the first target region. In some examples, the first target region includes a portion of a vena cava of the subject, a portion of the superior vena cava of the subject, a portion of an inferior vena cava of the subject, or a portion of an adjacent vessel to the vena cava, the superior vena cava, or the inferior vena cava. In some examples, the process 1100 may capture RAP measurements at the first target region while simultaneously (or serially) capturing SVC pressure associated with intracranial venous pressure at the second target region. For example, the process 1100 may be performed to modulate a volume of blood flowing from the blood vessel into a right atrium to decrease right atrial pressure and/or SVC pressure associated with intracranial venous pressure of the subject and may modulate the volume of blood based to ensure neither pressure exceeds predefined pressure thresholds for RAP and SVC pressure. The process 1100 may be used for blood flow regulation in the SVC or the IVC, but can additionally, or alternatively, be used for any suitable applications, clinical or otherwise. In general, process 1100 may be used with any of the devices and/or systems described herein. For example, a processor 808 may carry out the steps of process 1100. In some examples, the process 1100 may take place on the occlusion devices described herein and assessments for triggering process 1100 may take place on an external computing device that is communicatively coupled to the occlusion device performing process 1100. In some examples, such assessments for triggering process 1100 may take place on the occlusion devices described herein without accessing external computing devices.

As an example, the device used with process 1100 may include device 100 having an expandable frame with a proximal end and a distal end and a longitudinal axis extending therethrough, an occlusion element with an inflow end and an outflow end. The inflow end may be at least partially installed within the distal end of the expandable frame, and the outflow end may be coupled to an end effector, as described elsewhere herein. The end effector may radially collapse at the outflow end and toward a central axis of the expandable frame, or radially expand away from the central axis of the expandable frame, in response to an actuation of a control wire coupled to a portion of the occlusion element.

In some examples, the device utilized in process 1100 includes an expandable frame including a proximal end and a distal end and a longitudinal axis extending therethrough and a membrane including an inflow end and an outflow end. The outflow end may be coupled to a plurality of elongate support members arranged radially around an outer surface of the membrane and extending substantially parallel to the longitudinal axis.

At block 1102, the process 1100 includes providing an implantable device implanted in the blood vessel. For example, a vessel occlusion device (e.g., device 100) may be introduced at a site in a blood vessel of a subject. Since the devices described herein may be partially or fully housed by a frame (e.g., a stent), the frame housing of the device may be introduced to a vessel or tissue site using a delivery system. In a coronary procedure, a catheter tip and/or catheter may be configured to pass from the right atrium into the coronary sinus to implant the device. For access to the venous circulation, for example, a catheter tip and/or catheter may be configured to pass from the radial artery into the superior vena cava to implant the device into a portion of the superior vena cava. Further, for central venous access, a catheter tip and/or catheter may be configured to pass from the femoral vein into the inferior vena cava to implant the device into a portion of the inferior vena cava.

At block 1104, the process 1100 includes monitoring, by at least one processor, pressure in the blood vessel. For example, the device 100 may be programmed to monitor a first blood pressure with a first sensor (e.g., sensor 140) at a first target region and monitor a second blood pressure with a second sensor (e.g., sensor 142) at a second target region. In some examples, the first target region is a portion of a vena cava of the subject, a portion of an SVC of the subject, a portion of an IVC of the subject, or a portion of an adjacent vessel to the vena cava, the SVC, or the IVC. In some examples, the second target region is upstream in the (SVC or the IVC) from the first target region. For example, the second target region may be upstream of the SVC-RA junction of the heart.

In some examples, the monitoring may include monitoring, by the sensor 140, a rate of increase in the first pressure over a first time period, and/or monitoring, by the sensor 142, a rate of increase in the second pressure over the first time period. In response to output from the monitoring, the processor 808 may trigger actuation of the occlusion device. For example, the device 100 may actuate an end effector (e.g., flap, membrane 112, expandable member, etc.) of the implantable device (e.g., device 100) after the first time period based on the rate of increase in the first pressure or the rate of increase in the second pressure, as described elsewhere herein.

At block 1106, the process 1100 includes causing actuation of the implantable device to modulate the blood flow through the blood vessel in which the device is implanted (and/or one or more adjacent vessels) according to a first actuation cycle of the implantable device configured to maintain the first pressure within the first predefined pressure range. For example, the device 100 may be actuated to modulate the blood flow according to a first predefined actuation cycle in response to detecting, based on the monitoring, when the first pressure (e.g., RAP) in the blood vessel is above a first predefined pressure range. An example first predefined pressure range may be about 8 mmHg to about 10 mmHg.

At block 1108, the process 1100 includes causing switching of the implantable device to a second actuation cycle to alter (e.g., change, increase, decrease, modify, etc.) the modulation of the blood flow through the blood vessel. For example, the device 100 may be actuated to modulate the blood flow according to a second predefined actuation cycle in response to detecting, based on the monitoring, when a second pressure (e.g., SVC upstream pressure) that is upstream of the first pressure is above a second predefined pressure threshold. An example second pressure threshold may be at or above about 25 mmHg. Switching from the first actuation cycle to a second actuation cycle may include reducing the occlusion amount being performed by device 100 to relieve or reduce the second pressure.

At block 1110, the process 1100 includes causing reversion of the implantable device to the first actuation cycle when the second pressure is detected to be within the second predefined pressure range. For example, the device 100 may be switched from the first actuation cycle to the second actuation cycle when the sensor 140 and/or sensor 142 detect that the second pressure (e.g., SVC upstream pressure) is within a range and thus the subject is not in danger of increased intracranial venous pressure at the detection time. Accordingly, the device 100 may actuate a recovery cycle with little to no occlusion occurring in the vessel or the device 100 may actuate another occlusion cycle to relieve additional first pressure (e.g., RAP) that may have increased during performance of the second actuation cycle.

In some examples, the first actuation cycle is performed for a first time period and the second actuation cycle is performed for a second time period to maintain the first pressure within the first predefined pressure range and the second pressure within the second predefined pressure range. The first time period may be about 2 minutes to about 1 hour of actuation, as described else herein. The second time period may be about 2 minutes to about 1 hour. In some examples, the first and second time periods may be programmed to ensure that the occlusion device (e.g., an end effector of the occlusion device) does not remain stagnant. In this example, the end effector may oscillate between open and partially occluded. In some examples, the occlusion device may be periodically flushed to clear upstream blood stasis.

In some examples, the first actuation cycle reduces blood flow through the blood vessel to reduce the first pressure and/or reduce at least a portion of the second pressure. In some examples, the second actuation cycle increases blood flow through the blood vessel to reduce at least a portion of the second pressure.

In some examples, the first actuation cycle may be modified based on the first pressure (e.g., RAP) in the blood vessel, the second pressure (e.g., SVC upstream pressure) in the blood vessel, and one or more of: a determined cardiac pulsatility measured by the device 100, a respiratory effect detected by the device 100, a physiological effect detected by the device 100, and an activity exertion level detected by the device 100.

In some examples, monitoring the pressure further includes communicatively coupling the implantable device (e.g., device 100) to a first external computing device (e.g., device 805a and/or a second external computing device (e.g., device 805b) and causing transmission of output data to the second external computing device. The output data may correspond to the first pressure and the second pressure. For example, the output data may be sensor 140 output and/or sensor 142 output corresponding to detected pressure measurements of the subject. The monitoring may further include having the device 100 (e.g., processor 808) receive, from the second external computing device and based on the output data, health-based instructions for the subject to perform and/or for the device 100 to execute. The health-based instructions may also be triggered for display on the first external computing device, such as a smartphone, tablet, or other computing device for receiving health-based instructions associated with device 100 and the subject in which device 100 is implanted. In some examples, the health-based instructions may include one or more of: instructions to titrate medication, instructions to visit a clinic, instructions to deliver rescue therapy, and instructions to perform physical movements, or the like.

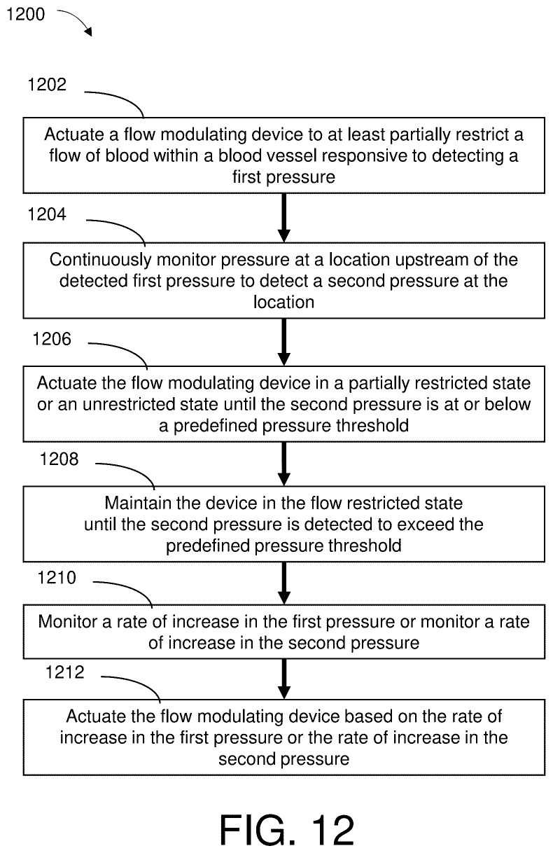

FIG. 12 illustrates a flow diagram of an example process 1200 for monitoring pressure and modulating blood flow through one or more blood vessels. In particular, the process 1200 may include monitoring pressure in a subject in which a flow modulation device is implanted, as described in detail for the process 1100 of FIG. 11.

The process 1200 may be used for blood flow regulation in the SVC or the IVC, but can additionally, or alternatively, be used for any suitable applications, clinical or otherwise. In general, process 1200 may be used with any of the devices and/or systems described herein. For example, a processor 808 may carry out the steps of process 1200 on device 100. In some examples, the process 1200 may take place on the occlusion devices described herein and assessments for triggering process 1200 may take place on an external computing device that is communicatively coupled to the occlusion device performing process 1200. In some examples, such assessments for triggering process 1200 may take place on the occlusion devices described herein without accessing external computing devices.

The device used in process 1200 may include an expandable frame having a proximal end and a distal end and a longitudinal axis extending therethrough, an occlusion element with an inflow end and an outflow end. The inflow end may be at least partially installed within the distal end of the expandable frame, and the outflow end may be coupled to an end effector, as described elsewhere herein. The end effector may radially collapse at the outflow end and toward a central axis of the expandable frame, or radially expand away from the central axis of the expandable frame, in response to an actuation of a control wire coupled to a portion of the occlusion element.

In some examples, the device utilized in process 1200 includes an expandable frame including a proximal end and a distal end and a longitudinal axis extending therethrough and a membrane including an inflow end and an outflow end. The outflow end may be coupled to a plurality of elongate support members arranged radially around an outer surface of the membrane and extending substantially parallel to the longitudinal axis.

At block 1202, the process 1200 includes actuating a flow modulating device at least partially positioned in a blood vessel in a flow restriction state to at least partially restrict a flow of blood within the blood vessel. For example, the device 100 may actuate an end effector (e.g., flap, membrane 112, expandable member, valve, etc.) to begin a flow restriction state to at least partially restrict the flow of blood in response to detecting a first pressure in the blood vessel. In some examples, the blood vessel is the SVC, the first pressure is a RAP, and the second pressure is SVC pressure indicating a level of intracranial venous pressure upstream of the first pressure.

At block 1204, the process 1200 includes continuously monitoring pressure at a location upstream of the detected first pressure in the blood vessel. For example, device 100 may use a processor (e.g., processor 808) and the first sensor 140 to monitor the RAP pressure and the second sensor 142 to monitor as second pressure at the location upstream of the detected first pressure. The monitoring may be the basis for performing one or more of block 1206, block 1208, block 1210, and/or block 1212.

At block 1206, the process 1200 includes actuating the flow modulating device to a partially restricted state or an unrestricted state to at least partially release the restricted flow of blood in response to determining that the second pressure is above a predefined pressure threshold, as described elsewhere herein.

In response to determining that the second pressure is at or below the predefined pressure threshold, the process 1200 may include maintaining the device in the flow restriction state until the second pressure is detected to exceed the predefined pressure threshold or upon completion of a predefined cycle configured for the flow modulating device, at block 1208; monitoring a rate of increase in the first pressure or monitoring a rate of increase in the second pressure, at block 1210; and actuating the flow modulating device based on the rate of increase in the first pressure or the rate of increase in the second pressure, at block 1212.

In some examples, the predefined cycle is an occlusion cycle that is selected based at least in part on an initial configuration of the flow modulating device and modified to increase or decrease modulation of the blood flow based on the monitoring. For example, the processor 808 may be programmed to determine which occlusion cycle to select and execute based on one or more: initial configurations of the device 100, initial configurations associated with the subject or condition of the subject, initial or ongoing measurements of pressure detected for the subject, and the like.

In some examples, modifying the predefined cycle may be further based on the first pressure, the second pressure, and one or more of a determined cardiac pulsatility of the subject measured by the implantable device, a respiratory effect of the subject detected by the implantable device, a physiological effect detected by the implantable device, and/or an activity exertion level of the subject detected by the implantable device.

In some examples, monitoring one or more outputs from the first sensor and the second sensor may include actuating at least one control wire (e.g., control element 124) coupled to a portion of the membrane (e.g., membrane 112) and configured to control the membrane based on the monitoring of the one or more outputs.

In some examples, actuating the flow modulating device includes triggering the at least one control wire to move and/or form the membrane in an unrestricted blood flow state, a partially restricted blood flow state, or a restricted blood flow state. In some examples, actuating the flow modulating device includes triggering the control wire to move and/or form the membrane into any state in between the restricted blood flow state and the unrestricted blood flow state. An example unrestricted blood flow state may correspond to the membrane 112 being triggered to radially expand away from a central axis of the expandable frame to allow blood flow through the blood vessel. An example restricted blood flow state may correspond to the membrane 112 being triggered to radially collapse toward the central axis of the expandable frame to reduce blood flow through the blood vessel. An example partially restricted blood flow state may correspond to triggering the membrane 112 to radially collapse or radially expand to partially occlude blood flow through the blood vessel. For example, the membrane 112 may be adjustable to a plurality of positions between expanded and collapsed. Example positions may include at least an expanded position to allow blood flow through the blood vessel, a partially expanded position to partially occlude the blood vessel, and a collapsed position to block the outflow end to occlude the blood vessel.

In some examples, the process 1100 and/or process 1200 may be a method of treatment for modulating blood flow in a superior vena cava in a subject having chronic kidney disease and/or chronic heart failure and/or intracranial venous pressure.

The method of treatment may include detecting, by the device, an anomalous event (or several events) associated with the blood vessel and actuating the device 100, for example, to trigger modulation of a flow of blood within the blood vessel at one or more sites within or substantially adjacent to a portion of the device 100.

In one non-limiting example, the devices described herein may perform a method of treatment for reducing right atrial pressure for a first target region in a blood vessel of a heart of a subject and reducing intracranial pressure at a second target region associated with the subject. The method of treatment may include introducing a device in the blood vessel and actuating, based on the monitoring of the outputs, the device to modulate a flow of blood within the blood vessel. In some examples, actuating the device may cause a partial occlusion of blood in the blood vessel. In some examples, modulating a volume of blood flowing from the blood vessel into a right atrium to decrease RAP at the first target region and decrease intracranial pressure at the second target region, as described elsewhere herein.

For example, the device may include a first sensor electrically coupled to the device and arranged to detect a first pressure in a blood vessel. The device may further include an optional second sensor electrically coupled to the device and arranged to detect a second pressure in the blood vessel at a location upstream of the first sensor. The device may also include a processing module electrically coupled to the first sensor and the optional second sensor. The processing module (e.g., processor 808, system 800) may be arranged to monitor outputs from the first sensor and the second sensor. Such monitoring may trigger notifications, indications, and instructions for the occlusion device to analyze, display, and or execute.

In some examples, the method of treatment may include de-actuating the device to maintain or regain the flow of blood within the blood vessel. In some examples, the first target region includes a portion of the SVC of the subject, or a portion of an IVC of the subject. In some examples, the blood vessel is the SVC and the device is implanted in a portion of the SVC of a subject having chronic kidney disease and/or chronic heart failure.

In some examples, the device utilized in the method treatment described herein includes an expandable frame including a proximal end and a distal end and a longitudinal axis extending therethrough and an occlusion element including an inflow end and an outflow end. The inflow end may be at least partially installed within the distal end of the expandable frame, and the outflow end may be coupled to an end effector arranged around an outer surface of the membrane and extending substantially parallel to the longitudinal axis.

In some examples, the device utilized in the methods described herein includes an expandable frame including a proximal end and a distal end and a longitudinal axis extending therethrough and a membrane including an inflow end and an outflow. The outflow end may be coupled to a plurality of elongate support members arranged radially around an outer surface of the membrane and extending substantially parallel to the longitudinal axis. One or more of the elongate support members may radially collapse at the outflow end and toward a central axis of the expandable frame, or radially expand away from the central axis of the expandable frame, in response to an actuation of a control wire coupled to a portion of the membrane/device.

Further, one or more sensors may be used in conjunction with any of the devices and systems herein to measure one or more physical characteristics of a subject having one of the devices implanted. For example, it may be beneficial to measure whether the subject is standing, sitting, or laying. In addition, the pressure thresholds for activating the device may be influenced by the activity of the subject. For example, it may be beneficial to realize the subject is exercising, as this would elevate pressures and may cause an adjustment in pressure thresholds. Characteristics described above may be measured by a pressure sensor in blood vessels of other portions of the body, a gyroscopic sensor for changes in angular position, an accelerometer for changes in acceleration, a heart rate sensor, a sensor measuring a size of a blood vessel, or any other sensors for measuring physical characteristics. The described characteristics, individually or in combination, may be received by a processor and processed to cause changes in valve/membrane/occlusion device position (using an actuating device) based on the sensed characteristics.

### Example Implantation of Flow Modulating Devices

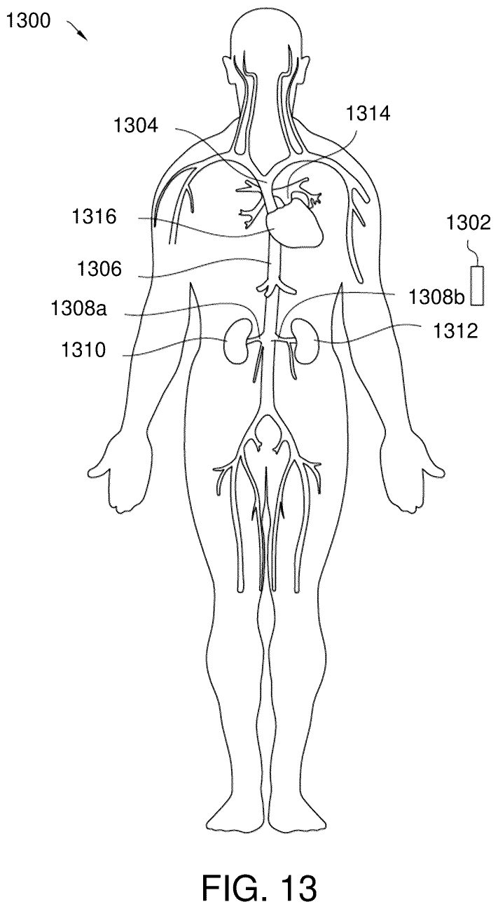

FIG. 13 illustrates a schematic representation of portions of a subject 1300. The flow modulating devices described herein of FIGS. 1A-10 (represented in FIG. 13 by device 1302) may be introduced (e.g., implanted) in vasculature of the body. In general, the device 1302 may represent any of the flow modulating devices described herein (e.g., device 100, device 804, system 800, etc.) and may include the same or similar functionality and/or structures. In some examples, the device 1302 may be implanted in or near to a portion of the SVC 1304. In some examples, the device 1302 may be implanted in or near to a portion of the IVC 1306. The subject 1300 is illustrated with a representation of a portion of the vasculature system to generally illustrate the SVC 1304 and the IVC 1306 within the subject 1300. However, it is to be understood that no dimensions or relative sizes of components may be inferred from the relative sizes and dimensions of elements in the figures.

The subject 1300 includes a number of vessels and organs that may circulate blood throughout the body. For example, renal veins 1308a and 1308b drain blood from respective right kidney 1310 and left kidney 1312. Renal veins 1308a and 1308b connect to the IVC 1306. Blood from the aorta 1314 flows to the IVC 1306. Blood travels from the aorta 1314 to the abdominal organs including the stomach (not shown), liver (not shown), spleen (not shown), pancreas (not shown), large intestines (not shown), and small intestine (not shown). Following processing of the blood by the liver, blood collects in the central vein. Blood from these central veins converges in the hepatic veins (not shown) which exit the liver and empty into the IVC 1306 to be distributed to the rest of the body.

Portions of the above-recited blood circulating vessels and/or organs may be involved in splanchnic venous circulation that includes blood flow originating from the celiac, superior mesenteric, and inferior mesenteric arteries to the abdominal organs. The splanchnic venous circulation may act as a blood reservoir that can support the need for increased stressed blood volume during periods of elevated sympathetic tone, such as during exertion, to support increased cardiac output and vasodilation of peripheral vessels supporting active muscles.

Heart failure subjects can have multiple comorbidities that cause excessive congestion or accumulation of blood volume in the splanchnic venous circulation and/or resulting increases in intracranial pressure based on the accumulation of blood volume. The excessive congestion or accumulation causes excess load on the heart, over-reactive fight or flight responses, poor oral medication absorption, etc. Example comorbidities can include chronic kidney disease, chronotropic incompetence, inability to increase stroke volume, and/or peripheral microvascular dysfunction. This can lead to venous congestion and/or abrupt rises in central venous pressure, pulmonary artery pressure, and/or pulmonary capillary wedge pressure. To alleviate such pressures, the blood reserves within the blood reservoir described above can be used to support the need for increased stressed blood volume during periods of elevated sympathetic tone. The flow modulating devices described herein may be used to ensure that such blood reserves within the blood reservoir can be utilized. For example, because blood flow from the splanchnic venous circulation is directed through hepatic veins and into the IVC 1306, devices (as described herein) may be placed into the IVC 1306 to limit blood flow to allow the splanchnic venous circulation to expand with increased blood volume. This may also allow the body to accumulate blood volume in the splanchnic venous circulation, which can maximize the downstream drop of pressure relative to upstream increase of pressure. Similarly, devices (as described herein) may be placed into the SVC 1308 to monitor pressures and/or limit blood flow to allow the reservoir to expand with increased blood volume. Furthermore, the flow modulating devices described herein may be placed in either the IVC 1306 and/or SVC 1308 to monitor and/or alleviate pressure in the right side of the atrium of the heart 1316 and/or regulate renal venous pressure and kidney function. Another example positioning of a flow modulating device may be in the IVC below the renal veins. This positioning may have a similar effect as the SVC location, as it may allow the flow modulating device to maintain renal venous pressure, which can correlate with sustained renal function and diuresis.

In some examples, the flow modulating device 1302 (representing the devices described herein) may be used as a method of treatment to treat any combination of heart failure, chronic kidney disease, chronotropic incompetence, inability to increase stroke volume, and/or peripheral microvascular dysfunction. In addition, the flow modulating device 1302 may be used as a method of treatment to regulate pressure in the right atrium of the heart and/or regulate intracranial venous pressure. Further, the flow modulating device 1302 may be used as a method of treatment to improve function of the kidneys in subjects having reduced kidney function due to pressure in the venous system.

For example, any of the implantable devices and/or systems described herein may be configured to modulate a volume of blood flowing from a superior vena cava into a right atrium to decrease right atrial pressure and/or to decrease intracranial venous pressure.

Further for example, any of the implantable devices and/or systems described herein may be used to perform a method including restricting blood flow within a blood vessel.

Still further for example, any of the implantable devices and/or systems described herein may be used to perform a method of treatment for a subject having one or both of: congestive heart failure or chronic kidney disease. The method may include restricting blood flow within the blood vessel.

As used herein, the term “active” with respect to blood flow management may represent operations carried out by the devices described herein using power or controller induced movement. For example, actively moving a portion of the devices described herein may include the use of battery power, wall outlet power, magnetic field induction, electromagnetic field induction, magnetic polarization, a piston-based system, a valve based system (e.g., with a manifold), hydraulics, pneumatics, optical actuators, thermal actuators, and/or other actuator using electrical or inductive power.

In some examples, an active control mechanism may include a microcontroller and/or a power source implanted with or integrated with the flow management device. Alternatively, or additionally, an active control mechanism can include a microcontroller and/or a power source in a remote control device, external to the body, or in an implanted remote device (e.g., subcutaneously, intravascularly, etc.), for example. The remote control device may be in wireless communication with the implanted device or connected to the implanted device through one or more leads.

In any of the embodiments described herein, an active mechanism may include a pump fluidly connected to a reservoir; a chamber having a first portion and a second portion; a manifold fluidly connected to the pump, the reservoir, and the chamber; and a piston coupled to a control element of a flow modulating device. The manifold may include at least one port that fluidly connects the reservoir to the first portion of the chamber. The piston can move between a restricted blood flow position and an unrestricted blood flow position within the chamber or any position therebetween for intermediate blood flow restriction positions. For example, the piston may move to the restricted blood flow position when a fluid flows from (or is pumped from) the reservoir through the manifold into the first portion of the chamber. The piston can return to the unrestricted blood flow position when the fluid is evacuated from the first portion of the chamber. In some examples, the manifold is fluidly connected to a second portion of the chamber through a second port. In such embodiments, the piston can move to the unrestricted blood flow position when the fluid enters the second port from the reservoir through the manifold, thereby causing the valve of the flow modulating device to move to the unrestricted blood flow state. In some examples, the fluid is evacuated from the second portion of the chamber through the second port when the piston is in the restricted blood flow position. In some implementations, the at least one port further fluidly connects the first portion of the chamber to the pump through the manifold. For example, the at least first port is fluidly connected to the pump through the manifold to evacuate the fluid from the first portion of the chamber thereby moving the piston to the unrestricted blood flow position. In some examples, the piston is a spring-based piston. For example, the spring-based piston can automatically return to the unrestricted blood flow position when the fluid is evacuated from the first portion of the chamber.

In any of the embodiments described herein, an active mechanism may include an actuator (e.g., a linear actuator) coupled to a control element of the flow management device. The linear actuator tensions the control element to position the valve of the flow management device in a restricted blood flow state. Alternatively, the linear actuator releases tension in the control element to position a valve, a membrane, or other material in an unrestricted blood flow state. The tensioning and releasing of tension on the control element may be based on a predefined set of parameters or based on a sensed attribute of the blood vessel in which the flow management device is implanted. For example, the sensed attribute may be sensed by a sensor. The sensor may be coupled to the flow management device, a remote control device, or otherwise in wireless or electrical communication with a flow management system. The sensor can be a strain gauge, a piezoelectric sensor, a capacitance sensor, or a vacuum pressure sensor, such that the sensor senses a pressure in the blood vessel.

In any of the embodiments described herein, the linear actuator is an electromechanical linear actuator having a first magnet that, when caused to rotate by another magnet or actuator, causes a nut to rotate on a lead screw, the nut being coupled to the control element. A second magnet in a control device may cause rotation of the first magnet, for example by changing its magnetic field pole direction. In some examples, a repeater magnet (with or without its own power source) is positioned between the first magnet and the second magnet, for example in cases where the first magnet is beyond a threshold distance from the second magnet.

In any of the embodiments described herein, the linear actuator is a pneumatic linear actuator having a piston coupled to the control element. Injecting compressed gas moves the piston to tension the control element to move the valve into a restricted blood flow state and venting the compressed gas releases tension in the control element to move the valve to an unrestricted blood flow state.

In any of the embodiments described herein, the linear actuator is a hydraulic linear actuator having a piston coupled to the control element. Injecting liquid moves the piston to tension the control element to move the valve into a restricted blood flow state and venting the liquid releases tension in the control element to move the valve to an unrestricted blood flow state.

In any of the embodiments described herein, the linear actuator is a thermal linear actuator having a piston coupled to the control element. For example, decreasing a temperature of a thermal sensitive fluid (e.g., via a heat source, changes in body temperature, etc.) causes the piston to compress the fluid to tension the control element to move the valve into the restricted blood flow state. Alternatively, increasing the temperature of the thermal sensitive fluid causes the piston to decompress the fluid to release tension in the control element to move the valve to the unrestricted blood flow state.

As used herein, the term “passive” with respect to blood flow management may represent operations carried out by the devices described herein using passively induced movement. For example, passively moving a portion of the devices described herein may include the use of manual pull wires (e.g., sutures, actuation wires/cords, etc.), anatomy responses (e.g., changes in vessel inner diameter, intra-vessel pressure, etc.), blood movement, or the like.

Any of the implantable or flow modulating devices described herein may be coated with a polymer (e.g., silicones, poly(urethanes), poly(acrylates), or copolymers such as poly(ethylene vinyl acetate), a drug (e.g., heparin, pro-endothelialization drugs, anti-thrombogenic drug, etc.), a textile (e.g., woven, knitted, nonwoven, or braided), tissue (e.g., bovine pericardium, equine pericardium, porcine vena cava, etc.), or a combination thereof. Woven and knitted fabrics may be made from poly(ethylene terephthalate), while the nonwoven fabrics may be made from expanded poly(tetrafluoroethylene). Some textiles may also or alternatively include silk or silk-based materials.

Further, any of the pull wires, sutures, frames/stents, or actuation wires described herein may include silk, silk-based materials, nylon, synthetic polymer materials (e.g., silicone, polydioxanone, polyglycolic acid, polyglyconate, polylactic acid, etc.), natural materials (e.g., purified catgut, collagen, sheep intestines, cow intestines, etc.), metal (e.g., Nitinol®, palladium, gold and their alloys, etc.), or a combination thereof.

The flow modulating devices described herein may be part of (or installed within) a stent. The stent may represent a frame or outer frame that provides a support structure for the flow modulating devices when the stent is implanted into a blood vessel. The frame/outer frame may be a self-expanding frame or a balloon-expandable frame. In general, any type of stent may be used with the flow modulating devices. Example stents may include, but are not limited to, bare metal stents, coated stents, drug-eluting stents, biodegradable stents, balloon expandable stents, and self-expandable stents.

The stents described herein may be configured to house all or a portion of the flow modulating devices described herein. Such stents may include an assembly with strut members interconnected by joints that form a series of linked mechanisms that result in a hollow tube-shaped element. The stents may be positioned and/or repositioned within a blood vessel to introduce or remove flow modulating devices or device members including, but not limited, to valving, control elements, balloons, flexible members, rigid members, adjustment mechanisms, sensors, coils, wires, and/or magnets. One or more of such device members may be actuated to modify stent shape (or device member shape) for purposes of modifying a flow of fluid through the vessel associated with the implanted stent. Moreover, the stents described herein may partially or fully surround a flow modulating device. For example, a stent or stent portion may surround a portion of a flow modulating device to ensure the device remains in a specified position in a blood vessel. In some examples, the stent surrounds the flow modulating device entirely. In some examples, the stent surrounds the flow modulating device and further continues beyond one or both ends of the device.

The stents described herein may include an outer frame. The outer frame may have a form and structure that varies. For example, the strut members and/or articulated joints may form a mesh-like structure. The strut members may be interconnected in such a way as to form a shaped pattern of cells. For example, any number of strut members may form a ring of the stent such that the strut members are connected by any number of crowns. Any number of rings may form a body of the stent, and the rings may be connected by any number of bridges. Example cell shapes may include, but are not limited to diamond, square, rectangle, triangle, oval, ganglion, or any combination thereof. In some examples, the cells may be evenly shaped and distributed from a first end of the stent to a second end of the stent. In some examples, the cells may include a number of strut members interconnected in such a way that when the stent expands radially, one or more of the cells become longitudinally shorter. Similarly, when the stent constricts radially, one or more of the cells become longitudinally longer.

Constricting portions of the stents described herein may result in an outer frame woven tighter than other portions of the stent that are not constricted. The constriction may push against one or more portions of the flow modulating devices described herein to narrow a pathway through the frame or outer frame and/or to trigger the flow modulating device to begin or end constriction. Similarly, expanding portions of the stents described herein may result in an outer frame woven looser than other portions of the stent that are not expanded. The expansion may release one or more portions of the flow modulating devices described herein to widen a pathway through the frame or outer frame and/or to trigger the flow modulating device to begin or end constriction.

The flow modulating devices described herein may be introduced to a vessel or tissue site using a delivery system. For example, such delivery systems may be used to position catheter tips and/or catheters in various portions of a target vasculature. A delivery system may include a delivery catheter having a pusherwire or the like disposed therein. The pusherwire may be configured to deploy any of the devices described herein, for example by urging the device out of a distal end of the catheter and either actively expanding the device or allowing the device to passively expand once it is no longer constrained by a lumen of the catheter. Any of the devices described herein may be crimped or otherwise compressed such that a cross-sectional area of the device is sized and/or shaped to be delivered through a lumen of a catheter. In some examples, the crimped or compressed device may be transferred to the delivery system using a transfer sheath, or the like. A delivery system can access the vasculature through an access site, such as a radial artery, brachial artery, internal jugular vein, common femoral vein, subclavian veins, or the like.

For example, in a coronary procedure, a catheter tip and/or catheter may be configured to pass from the right atrium into the coronary sinus. For access to the venous circulation, for example, a catheter tip and/or catheter may be configured to pass from the radial artery into the superior vena cava. Further, for central venous access, a catheter tip and/or catheter may be configured to pass from the femoral vein into the inferior vena cava.

In some examples, the delivery system may include a trocar or other suitable delivery device used for implanting devices subcutaneously, for example control devices for controlling activation of any of the flow modulating devices described herein. As described elsewhere herein, various control systems may include an implanted remote device that is configured to transmit control signals to a flow modulating device disposed in the vasculature. The control signals may include signals transmitted wirelessly, through a wired connection (e.g., leads), or via magnetic field induction, electromagnetic field induction, or magnetic polarization.

However, it will be understood that the delivery system can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels (e.g., superior vena cava, inferior vena cava, renal artery, renal vein, etc.), and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device including an inner lumen configured to slidably receive instrumentation, such as for positioning within an atrium, coronary sinus, superior vena cava, or inferior vena cava, including for example delivery catheters, cannulas, and/or trocars. It will be understood that other types of medical implant devices and/or procedures can be delivered to the coronary sinus, superior vena cava, inferior vena cava, etc. using a delivery system as described herein, including for example ablation procedures, drug delivery, and/or placement of actuator leads.

Described herein are various example medical implants and/or delivery methods. Some examples described herein may be used in combination and/or may be used independently.

Provided below is a list of examples, each of which may include aspects of any of the other examples disclosed herein. Furthermore, aspects of any example described above may be implemented in any of the numbered examples provided below.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Example 1. A method for modulating blood flow through a blood vessel, the method comprising: providing an implantable device implanted in the blood vessel; monitoring, by at least one processor, pressure in the blood vessel; in response to detecting, based on the monitoring, when a first pressure in the blood vessel is above a first predefined pressure range, causing actuation of the implantable device to modulate the blood flow through the blood vessel or an adjacent blood vessel according to a first actuation cycle of the implantable device configured to maintain the first pressure within the first predefined pressure range; in response to detecting, based on the monitoring, when a second pressure upstream of the first pressure is above a second predefined pressure threshold, causing switching of the implantable device to a second actuation cycle to alter the modulation of the blood flow through the blood vessel; and causing reversion of the implantable device to the first actuation cycle when the second pressure is detected to be within the second predefined pressure range.

Example 2. The method as in any of the above examples and in particular example 1, wherein the first actuation cycle is performed for a first time period and the second actuation cycle is performed for a second time period to maintain the first pressure within the first predefined pressure range and the second pressure within the second predefined pressure range.

Example 3. The method as in any of the above examples and in particular example 2, wherein the first actuation cycle is configured to be modified based on the first pressure in the blood vessel, the second pressure in the blood vessel, and one or more of: a determined cardiac pulsatility measured by the implantable device; a respiratory effect detected by the implantable device; a physiological effect detected by the implantable device; and an activity exertion level detected by the implantable device.

Example 4. The method as in any of the above examples and in particular example 1, wherein the monitoring the pressure further comprises: communicatively coupling the implantable device to a first external computing device and a second external computing device; causing transmission of output data to the second external computing device, the output data corresponding to the first pressure and the second pressure; and receiving, from the second external computing device and based on the output data, health-based instructions, the health-based instructions being triggered for display on the first external computing device.

Example 5. The method as in any of the above examples and in particular example 4, wherein the detecting of the first pressure and the detecting of the second pressure is performed over a predefined time period; and the output data comprises a time-in-range calculation determined for the predefined time period, the time-in-range calculation comprising determining an amount of time in which the first pressure is within the first predefined pressure range and the second pressure is within the second predefined pressure range divided by the predefined time period.

Example 6. The method as in any of the above examples and in particular example 4, wherein the health-based instructions comprise one or more of: instructions to titrate medication, instructions to visit a clinic, instructions to deliver rescue therapy, and instructions to perform physical movements.

Example 7. The method as in any of the above examples and in particular example 1, wherein the monitoring further comprises: monitoring a rate of increase in the first pressure over a first time period; monitoring a rate of increase in the second pressure over the first time period; actuating a membrane of the implantable device after the first time period based on the rate of increase in the first pressure or the rate of increase in the second pressure.

Example 8. The method as in any of the above examples and in particular example 1, wherein the first actuation cycle reduces blood flow through the blood vessel or the adjacent blood vessel to reduce the first pressure and reduce the second pressure.

Example 9. The method as in any of the above examples and in particular example 1, wherein the second actuation cycle increases blood flow through the blood vessel to reduce at least a portion of the second pressure.

Example 10. An implantable device for dynamically modulating blood flow through a blood vessel, the implantable device comprising: an expandable frame comprising a proximal end and a distal end and a longitudinal axis extending therethrough; and an occlusion element comprising an inflow end and an outflow end, wherein the inflow end is at least partially installed within the distal end of the expandable frame, and the outflow end is coupled to an end effector configured to modulate the blood flow through the blood vessel; and a first sensor positioned at a distal end of the expandable frame and configured to detect a first pressure in the blood vessel; a second sensor positioned upstream of the first sensor and configured to detect a second pressure in the blood vessel at a location upstream of the first sensor; a processor electrically coupled to the first sensor and the second sensor, wherein the processor is configured to: monitor the first pressure and the second pressure sensed by the first and second sensors; and actuate the occlusion element to at least partially occlude the blood vessel at the end effector based on the monitored first pressure or the monitored second pressure.

Example 11. The implantable device as in any of the above examples and in particular example 10, wherein actuating the occlusion element reduces blood flow through the blood vessel or an adjacent blood vessel to reduce the first pressure and reduce the second pressure.

Example 12. The implantable device as in any of the above examples and in particular example 10, wherein actuating the occlusion element increases blood flow through the blood vessel or an adjacent blood vessel to reduce the second pressure.

Example 13. The implantable device as in any of the above examples and in particular example 10, wherein actuating the occlusion element is performed based on a determined elapsed time in which the monitored first pressure is within a predefined pressure range, the elapsed time being determined based at least in part on the monitored first pressure and the monitored second pressure.

Example 14. The implantable device as in any of the above examples and in particular example 10, wherein actuating the occlusion element is based on the monitoring, the actuating comprising: radially collapsing the occlusion element at the outflow end and toward a central axis of the expandable frame in response to detecting the first pressure is above a predefined pressure threshold; and radially expanding at least a portion of the occlusion element away from the central axis of the expandable frame in response to determining that the second pressure is increasing at or above a predefined rate.

Example 15. The implantable device as in any of the above examples and in particular example 10, wherein actuating the occlusion element to at least partially occlude the blood vessel comprises selecting an occlusion level for the implantable device according to the detected first pressure and occluding the blood vessel according to the selected occlusion level.

Example 16. The implantable device as in any of the above examples and in particular example 15, wherein the occlusion level is a percentage from about 0 percent occluded to about 100 percent occluded.

Example 17. The implantable device as in any of the above examples and in particular example 15, wherein the occlusion level is increased in response to detecting that the first pressure is above a predefined pressure range for a time exceeding a predefined time threshold.

Example 18. The implantable device as in any of the above examples and in particular example 17, wherein the occlusion level is decreased in response to detecting that the second pressure is above a second predefined pressure range at a time after the predefined time threshold.

Example 19. The implantable device as in any of the above examples and in particular example 18, wherein the second predefined pressure range for the second pressure comprises about 10 mmHg to about 25 mmHg.

Example 20. The implantable device as in any of the above examples and in particular example 18, wherein the second predefined pressure range for the second pressure comprises about 15 mmHg to about 20 mmHg.

Example 21. The implantable device as in any of the above examples and in particular example 17, wherein the predefined pressure range for the first pressure comprises about 2 mmHg to about 10 mmHg.

Example 22. The implantable device as in any of the above examples and in particular example 17, wherein the predefined pressure range for the first pressure comprises about 8 mmHg to about 10 mmHg.

Example 23. The implantable device as in any of the above examples and in particular example 15, wherein the occlusion level is selected to maintain a first predefined pressure range for the first pressure in the blood vessel and maintain a second predefined pressure range for the second pressure in the blood vessel for about 80 percent of a predefined cycle time associated with the monitoring.

Example 24. The implantable device as in any of the above examples and in particular example 10, wherein the monitoring of the first pressure and the second pressure further comprises: communicatively coupling the implantable device to a first external computing device and a second external computing device; transmitting, to the second external computing device, output data corresponding to the monitored first pressure and the monitored second pressure; and receiving, from the second external computing device and based on the transmitted output data, health-based instructions, the health-based instructions being triggered for display on the first external computing device.

Example 25. The implantable device as in any of the above examples and in particular example 24, wherein the health-based instructions comprise one or more of: instructions to titrate medication, instructions to visit a clinic, instructions to deliver rescue therapy, and instructions to perform physical movements.

Example 26. The implantable device as in any of the above examples and in particular example 10, wherein the monitoring of the first pressure and the second pressure comprises: monitoring a rate of increase in the first pressure over a first time period; monitoring a rate of increase in the second pressure over the first time period; actuating the occlusion element after the first time period based on the rate of increase in the first pressure or the rate of increase in the second pressure.

Example 27. The implantable device as in any of the above examples and in particular example 26, wherein actuating the occlusion element after the first time period comprises: actuating the occlusion element to occlude the blood vessel until the first pressure is determined to be within a first predefined pressure range; and modifying an occlusion level of the occlusion element in response to determining that the second pressure exceeds a second predefined pressure range.

Example 28. The implantable device as in any of the above examples and in particular example 27, wherein the second predefined pressure range comprises about 15 mmHg to about 20 mmHg.

Example 29. The implantable device as in any of the above examples and in particular example 27, wherein the first predefined pressure range comprises about 8 mmHg to about 10 mmHg.

Example 30. The implantable device as in any of the above examples and in particular example 26, wherein the monitoring of the first pressure and the monitoring of the second pressure is performed substantially continuously, the monitoring further comprising: in response to detecting, at a second time period, that the first pressure is at or below a predefined pressure threshold, generating an indication to adjust the occlusion element to a selected one of a plurality of positions between expanded and collapsed, the selected one of the plurality of positions selected based at least in part on the first pressure or the second pressure detected during the first time period.

Example 31. The implantable device as in any of the above examples and in particular example 10, wherein the monitoring of the first pressure and the second pressure comprises: detecting when the first pressure is above a first predefined pressure range; actuating the occlusion element to modulate the blood flow through the blood vessel according to a first actuation cycle for maintaining the first pressure within the first predefined pressure range; detecting when the second pressure is above a second predefined pressure threshold; switching the implantable device to a second actuation cycle to alter the modulation of the blood flow through the blood vessel; and reverting to the first actuation cycle when the second pressure is detected to be within the second predefined pressure range.

Example 32. The implantable device as in any of the above examples and in particular example 31, wherein the first actuation cycle is performed for a first time period and the second actuation cycle is performed for a second time period to maintain the first pressure within the first predefined pressure range and the second pressure within the second predefined pressure range.

Example 33. The implantable device as in any of the above examples and in particular example 32, wherein the first actuation cycle is configured to be modified based on the first pressure in the blood vessel, the second pressure in the blood vessel, and one or more of: a determined cardiac pulsatility measured by the implantable device when implanted into the blood vessel; a respiratory effect detected by the implantable device when implanted into the blood vessel; a physiological effect detected by the implantable device when implanted into the blood vessel; and an activity exertion level detected by the implantable device when implanted into the blood vessel.

Example 34. The implantable device as in any of the above examples and in particular example 10, wherein actuating the occlusion element comprises radially collapsing the end effector at the outflow end and toward a central axis of the expandable frame or radially expanding the end effector away from the central axis of the expandable frame.

Example 35. The implantable device as in any of the above examples and in particular example 34, wherein a range of collapsing or expanding is selected based on one or more of: a predefined occlusion profile, a predefined occlusion schedule, a differential between the first pressure and the second pressure, and a detected rate of increase in the second pressure.

Example 36. The implantable device as in any of the above examples and in particular example 10, wherein: the blood vessel is a superior vena cava; the first pressure is right atrial pressure; and the second pressure is superior vena cava pressure indicating a level of intracranial venous pressure of a subject implanted with the implantable device.

Example 37. The implantable device as in any of the above examples and in particular example 10, wherein the occlusion element is adjustable to a plurality of positions between expanded and collapsed, the plurality of positions including at least: an expanded position configured to allow the blood flow through the blood vessel; a partially expanded position configured to partially occlude the blood vessel; and a collapsed position configured to block the outflow end to occlude the blood vessel.

Example 38. The implantable device as in any of the above examples and in particular example 10, wherein the occlusion element is: substantially tubular-shaped with a substantially circular cross section; and adjustable to form a cinched portion at the outflow end, the cinching resulting in reversibly reducing or closing the circular cross section at the outflow end.

Example 39. The implantable device as in any of the above examples and in particular example 10, wherein the blood vessel comprises an inferior vena cava.

Example 40. The implantable device as in any of the above examples and in particular example 10, wherein actuating the occlusion element comprises triggering a control wire coupled to a portion of the occlusion element, and wherein actuating the control wire results in configuring the occlusion element in an unrestricted blood flow state or a restricted blood flow state, wherein: the unrestricted blood flow state corresponds to the end effector radially expanding away from a central axis of the expandable frame to allow blood flow through the blood vessel; and the restricted blood flow state corresponds to the end effector radially collapsing toward the central axis of the expandable frame to reduce blood flow through the blood vessel.

Example 41. The implantable device as in any of the above examples and in particular example 40, wherein: the occlusion element is a membrane, the end effector comprises a plurality of elongate support members arranged radially around an outer surface of the membrane and extending substantially parallel to the longitudinal axis, and the device further comprises: a plurality of eyelets, wherein each respective eyelet of the plurality of eyelets is coupled to a distal end of each corresponding elongate support member in the plurality of elongate support members, each eyelet being configured to receive a portion of the control wire threaded therethrough such that the actuation of the control wire reversibly cinches the membrane by bringing the plurality of eyelets together at the outflow end to occlude or partially occlude the blood vessel.

Example 42. The implantable device as in any of the above examples and in particular example 41, wherein the reversible cinching may be performed to: fully collapse the outflow end of the membrane resulting in occlusion of the blood vessel; or partially collapse the outflow end of the membrane resulting in a partial occlusion of the blood vessel.

Example 43. The implantable device as in any of the above examples and in particular example 10, further comprising: a power source comprising a battery or a wall outlet, wherein the power source is coupled to a control wire configured to cause the occlusion element to be configured in an unrestricted blood flow state or a restricted blood flow state; and an actuation device for actuating the implantable device, the actuation device comprising: an actuator coupled to a control wire of the implantable device, and a first magnet configured to induce rotation of the actuator; and a control device communicatively coupled to the actuator, wherein the control device comprises a second magnet configured to generate a changing magnetic field pole direction to cause rotation of the first magnet.

Example 44. The implantable device as in any of the above examples and in particular example 43, wherein: the actuator is configured to send a first signal to the control wire to activate application of tension to the control wire; and the actuator is configured to send a second signal to the control wire to activate release of the tension from the control wire.

Example 45. The implantable device as in any of the above examples and in particular example 43, wherein the actuator is a magnetically driven actuator.

Example 46. The implantable device as in any of the above examples and in particular example 43, wherein the control device is implanted.

Example 47. The implantable device as in any of the above examples and in particular example 43, wherein the control device is implanted subcutaneously.

Example 48. The implantable device as in any of the above examples and in particular example 43, wherein the control device is disposed external to a body of a user associated with the implantable device.

Example 49. The implantable device as in any of the above examples and in particular example 10, wherein the implantable device is configured to modulate a volume of blood flowing from a superior vena cava into a right atrium to decrease right atrial pressure and to modulate intracranial venous pressure.

Example 50. A method for monitoring pressure in a subject in which a flow modulation device is implanted, the method comprising: in response to detecting a first pressure in a blood vessel, actuating the flow modulating device at least partially positioned in the blood vessel to a flow restriction state to at least partially restrict a flow of blood within the blood vessel or an adjacent blood vessel; continuously monitoring pressure at a location upstream of the detected first pressure in the blood vessel, the monitoring comprising: detecting a second pressure at the location; in response to determining that the second pressure is above a predefined pressure threshold, actuating the flow modulating device to a partially restricted state or an unrestricted state to at least partially release the restricted flow of blood; and in response to determining that the second pressure is at or below the predefined pressure threshold: maintaining the device in the flow restriction state until the second pressure is detected to exceed the predefined pressure threshold or upon completion of a predefined cycle configured for the flow modulating device; monitoring a rate of increase in the first pressure or monitoring a rate of increase in the second pressure; and actuating the flow modulating device based on the rate of increase in the first pressure or the rate of increase in the second pressure.

Example 51. The method as in any of the above examples and in particular example 50, wherein: the blood vessel is a superior vena cava; the first pressure is right atrial pressure; and the second pressure is superior vena cava pressure indicating a level of intracranial venous pressure.

Example 52. The method as in any of the above examples and in particular example 50, wherein: the blood vessel is the vena cava; the first pressure is right atrial pressure; and the second pressure is vena cava pressure indicating a level of intracranial venous pressure.

Example 53. The method as in any of the above examples and in particular example 50, wherein the predefined cycle is: selected based at least in part on an initial configuration of the flow modulating device; and modified to increase or decrease modulation of the blood flow based on the monitoring.

Example 54. The method as in any of the above examples and in particular example 53, wherein modifying the predefined cycle is further based on the first pressure, the second pressure, and one or more of: a determined cardiac pulsatility measured by the implantable device; a respiratory effect detected by the implantable device; a physiological effect detected by the implantable device; and an activity exertion level detected by the implantable device.

Example 55. The method as in any of the above examples and in particular example 50, wherein the flow modulating device comprises: an expandable frame comprising a proximal end and a distal end and a longitudinal axis extending therethrough; and an occlusion element comprising an inflow end and an outflow end, wherein the inflow end is at least partially installed within the distal end of the expandable frame, and the outflow end is coupled to an end effector configured to modulate the blood flow through the blood vessel; a first sensor coupled to the expandable frame and configured to detect the first pressure in the blood vessel; and a second sensor positioned upstream of the first sensor and configured to detect the second pressure in the blood vessel at a location upstream of the first sensor.

Example 56. The method as in any of the above examples and in particular example 55, wherein the flow modulating device further comprises at least one processor configured to perform operations including: monitoring one or more outputs from the first sensor and the second sensor; causing actuation of at least one control wire coupled to a portion of the occlusion element and configured to control the end effector based on the monitoring of the one or more outputs.

Example 57. The method as in any of the above examples and in particular example 56, wherein actuating the flow modulating device comprises: triggering the at least one control wire to configure the occlusion element in an unrestricted blood flow state, a partially restricted blood flow state, or a restricted blood flow state, wherein: the unrestricted blood flow state corresponds to the end effector radially expanding away from a central axis of the expandable frame to allow blood flow through the blood vessel; the restricted blood flow state corresponds to the end effector radially collapsing toward the central axis of the expandable frame to reduce blood flow through the blood vessel; and the partially restricted blood flow state corresponds to the end effector radially collapsing or radially expanding to partially occlude blood flow through the blood vessel.

Example 58. The method as in any of the above examples and in particular example 55, wherein the occlusion element is adjustable to a plurality of positions between expanded and collapsed, the plurality of positions including at least: an expanded position configured to allow the blood flow through the blood vessel; a partially expanded position configured to partially occlude the blood vessel; and a collapsed position configured to block the outflow end to occlude the blood vessel.

Example 59. An implantable system for alleviating pressure within a blood vessel, the system comprising: a device for modulating a flow of blood through the blood vessel; a first sensor electrically coupled to the device and configured to detect a first pressure in the blood vessel; a second sensor electrically coupled to the device and configured to detect a second pressure in the blood vessel at a location upstream of the first sensor; a processing module electrically coupled to the first sensor and the second sensor, wherein the processing module is configured to: monitor outputs from the first sensor and the second sensor; and actuate the device to perform blood flow modulation through the blood vessel based on the monitoring of the outputs.

Example 60. The implantable system as in any of the above examples and in particular example 59, wherein actuating the device reduces blood flow through the blood vessel or an adjacent blood vessel to reduce the first pressure and reduce the second pressure.

Example 61. The implantable system as in any of the above examples and in particular example 59, wherein actuating the device is further based on parameters for configuring actuation of the device.

Example 62. The implantable system as in any of the above examples and in particular example 61, wherein the parameters comprise one or more of: a clearance time parameter corresponding to an amount of time for clearing blood volume from one or more portions of the device; a peak occlusion parameter corresponding to a maximum occlusion capacity associated with operating the device; an activation time parameter corresponding to an amount of time the device is actively operating; a hold time parameter corresponding to an amount of time the occlusion is held at an occlusion level by the device; a cycle time parameter corresponding to an amount of time for completing an occlusion cycle; and a recovery time parameter corresponding to a minimum time between actuation performed by the device.

Example 63. The implantable system as in any of the above examples and in particular example 62, wherein the configurable parameters further comprise one or more of: a ramp up rate parameter for modulating the flow of blood through the blood vessel, the ramp up rate parameter corresponding to an amount of time to reach a selected percentage of occlusion by the device; a level of negligible resistance parameter corresponding to a level of occlusion determined to have negligible impact on resistance to blood flow through the device; and an agitation cycle parameter corresponding to an amount of time to allow the device to remain stationary before moving at least one portion of the device.

Example 64. The implantable system as in any of the above examples and in particular example 63, wherein the agitation cycle parameter is configured to schedule an agitation cycle to reduce stasis within or near to the implantable system.

Example 65. The implantable system as in any of the above examples and in particular example 59, wherein the device comprises a membrane having an inflow end and an outflow end, wherein the inflow end is at least partially installed within a portion of an expandable frame, and the outflow end is configured to expand and contract to modulate the flow of blood through the blood vessel.

Example 66. The implantable system as in any of the above examples and in particular example 65, wherein: the first sensor is positioned at a distal end of the expandable frame; and the second sensor is positioned at a proximal end of the expandable frame.

Example 67. The implantable system as in any of the above examples and in particular example 59, wherein: the blood vessel is a superior vena cava; the first pressure is right atrial pressure; and the second pressure is superior vena cava pressure indicating a level of intracranial venous pressure.

Example 68. The implantable system as in any of the above examples and in particular example 59, wherein: the blood vessel is a vena cava; the first pressure is right atrial pressure; and the second pressure is vena cava pressure indicating a level of intracranial venous pressure.

Example 69. A method of treatment for reducing right atrial pressure for a first target region in a blood vessel of a heart of a subject and reducing intracranial pressure at a second target region associated with the subject, the method comprising: introducing a device in the blood vessel, the device comprising: a first sensor electrically coupled to the device and configured to detect a first pressure in the blood vessel; a second sensor electrically coupled to the device and configured to detect a second pressure in the blood vessel at a location upstream of the first sensor; a processing module electrically coupled to the first sensor and the second sensor and configured to monitor outputs from the first sensor and the second sensor; actuating, based on the monitoring of the outputs, the device to modulate a flow of blood within the blood vessel or an adjacent blood vessel.

Example 70. The method as in any of the above examples and in particular example 69, further comprising: de-actuating the device to maintain or regain the flow of blood within the blood vessel or the adjacent blood vessel.

Example 71. The method as in any of the above examples and in particular example 69, wherein the first target region includes a portion of a vena cava of the subject, a portion of the superior vena cava of the subject, or a portion of an inferior vena cava of the subject.

Example 72. The method as in any of the above examples and in particular example 69, wherein actuating the device causes a partial occlusion of blood in the blood vessel.

Example 73. The method as in any of the above examples and in particular example 69, wherein the blood vessel is a superior vena cava and the device is configured to be implanted in a portion of the superior vena cava of a subject having chronic kidney disease and chronic heart failure; and the method further comprises modulating a volume of blood flowing from the blood vessel into a right atrium to decrease right atrial pressure at the first target region and decrease intracranial pressure at the second target region.

Example 74. The method as in any of the above examples and in particular example 69, wherein the device further comprises: an expandable frame comprising a proximal end and a distal end and a longitudinal axis extending therethrough; and an occlusion element comprising an inflow end and an outflow end, wherein the inflow end is at least partially installed within the distal end of the expandable frame, and the outflow end is coupled to an end effector configured to modulate the blood flow through the blood vessel, and wherein the end effector is configured to radially collapse at the outflow end and toward a central axis of the expandable frame, or radially expand away from the central axis of the expandable frame, in response to an actuation of a control wire coupled to a portion of the occlusion element.

Example 75. The method as in any of the above examples and in particular example 1, wherein the implantable device comprises: an expandable frame comprising a proximal end and a distal end and a longitudinal axis extending therethrough; and an occlusion element comprising an inflow end and an outflow end, wherein the inflow end is at least partially installed within the distal end of the expandable frame, and the outflow end is coupled to an end effector configured to modulate the blood flow through the blood vessel; a first sensor coupled to the expandable frame and configured to detect the first pressure in the blood vessel; and a second sensor positioned upstream of the first sensor and configured to detect the second pressure in the blood vessel at a location upstream of the first sensor.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It should be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

The systems and methods of the embodiments and variations described herein can be embodied and/or implemented at least in part as a machine configured to receive a computer-readable medium storing computer-readable instructions. The instructions may be executed by computer-executable components integrated or in communication with the system and one or more portions of the processor on or in communication with the control device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “projection” may include, and is contemplated to include, a plurality of projections. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method for modulating blood flow from a superior vena cava or an inferior vena cava to a right atrium, the method comprising:
providing an implantable device implanted at an implantation location in the superior vena cava or the inferior vena cava, wherein the implantable device is configured to occlude an amount of blood flowing through the implantable device and into the right atrium;
monitoring, by at least one processor, blood pressure upstream and downstream of the implantable device;
in response to detecting, based on the monitoring, when a first pressure downstream of the implantable device is above a first predefined pressure range, causing actuation of the implantable device to modulate the blood flow through the implantable device according to a first actuation cycle of the implantable device configured to maintain the first pressure within the first predefined pressure range;
in response to detecting, based on the monitoring, when a second pressure upstream of the implantable device is above a second predefined pressure threshold, causing switching of the implantable device to a second actuation cycle to alter the modulation of the blood flow through the implantable device; and
causing reversion of the implantable device to the first actuation cycle when the second pressure is detected to be within the second predefined pressure range.

2. The method of claim 1, wherein the first actuation cycle is performed for a first time period and the second actuation cycle is performed for a second time period to maintain the first pressure within the first predefined pressure range and the second pressure within the second predefined pressure range.

3. The method of claim 2, wherein the first actuation cycle is configured to be modified based on the first pressure, the second pressure, and one or more of:
a determined cardiac pulsatility measured by the implantable device;
a respiratory effect detected by the implantable device;
a physiological effect detected by the implantable device; and
an activity exertion level detected by the implantable device.

4. The method of claim 1, wherein the monitoring the pressure further comprises:
communicatively coupling the implantable device to a first external computing device and a second external computing device;
causing transmission of output data to the second external computing device, the output data corresponding to the first pressure and the second pressure; and
receiving, from the second external computing device and based on the output data, health-based instructions, the health-based instructions being triggered for display on the first external computing device.

5. The method of claim 4, wherein the detecting of the first pressure and the detecting of the second pressure is performed over a predefined time period; and
the output data comprises a time-in-range calculation determined for the predefined time period, the time-in-range calculation comprising determining an amount of time in which the first pressure is within the first predefined pressure range and the second pressure is within the second predefined pressure range divided by the predefined time period.

6. The method of claim 4, wherein the health-based instructions comprise one or more of: instructions to titrate medication, instructions to visit a clinic, instructions to deliver rescue therapy, and instructions to perform physical movements.

7. The method of claim 1, wherein the monitoring further comprises:
monitoring a rate of increase in the first pressure over a first time period;
monitoring a rate of increase in the second pressure over the first time period;
actuating an occlusion element of the implantable device after the first time period based on the rate of increase in the first pressure or the rate of increase in the second pressure.

8. The method of claim 1, wherein the first actuation cycle reduces blood flow through the implantable device to reduce the first pressure.

9. The method of claim 1, wherein the second actuation cycle increases blood flow through the implantable device to reduce at least a portion of the second pressure.

10. The method of claim 1, wherein the implantable device is configured to be implanted at the implantation location via catheterization and comprises:
an expandable frame comprising an inlet end and an outlet and a longitudinal axis extending therethrough; and
an occlusion element configured to move between an open state and an at least partially occluded state to modulate the blood flow through the implantable device;
a first sensor coupled to the expandable frame and configured to detect the first pressure; and
a second sensor positioned upstream of the first sensor and configured to detect the second pressure;
wherein the processor receives respective signals from the first sensor and the second sensor indicative of the first pressure and the second pressure, respectively, and actuate the occlusion element to move between the open state and the least partially occluded state, and vice versa.

11. A method for modulating blood flow to a right atrium with a flow modulating device implanted in a superior vena cava or an inferior vena cava, the method comprising:
in response to detecting a first pressure of blood flowing into the right atrium, actuating the flow modulating device to a flow restriction state to at least partially restrict a flow of blood into the right atrium;
continuously monitoring pressure of blood flowing into the flow modulating device at a location upstream of the detected first pressure, the monitoring comprising:
detecting a second pressure at the location;
in response to determining that the second pressure is above a predefined pressure threshold, actuating the flow modulating device to a partially restricted state or an unrestricted state to at least partially release the restricted flow of blood; and
in response to determining that the second pressure is at or below the predefined pressure threshold:
maintaining the device in the flow restriction state until the second pressure is detected to exceed the predefined pressure threshold or upon completion of a predefined cycle configured for the flow modulating device;
monitoring a rate of increase in the first pressure or monitoring a rate of increase in the second pressure; and
actuating the flow modulating device based on the rate of increase in the first pressure or the rate of increase in the second pressure.

12. The method of claim 11, wherein:
the flow modulating device is implanted in the superior vena cava;
the first pressure is right atrial pressure; and
the second pressure is superior vena cava pressure indicating a level of intracranial venous pressure.

13. The method of claim 11, wherein the predefined cycle is:
selected based at least in part on an initial configuration of the flow modulating device; and
modified to increase or decrease modulation of the blood flow based on the monitoring.

14. The method of claim 13, wherein modifying the predefined cycle is further based on the first pressure, the second pressure, and one or more of:
a determined cardiac pulsatility measured by the implantable device;
a respiratory effect detected by the implantable device;
a physiological effect detected by the implantable device; and
an activity exertion level detected by the implantable device.

15. The method of claim 11, wherein the flow modulating device comprises:
an expandable frame; and
an occlusion element coupled to the frame and configured to modulate the blood flow through the flow modulating device;
a first sensor coupled to the expandable frame and configured to detect the first pressure; and
a second sensor positioned upstream of the first sensor and configured to detect the second pressure.

16. The method of claim 15, wherein the flow modulating device further comprises at least one processor configured to perform operations including:
monitoring one or more outputs from the first sensor and the second sensor;
actuating the occlusion element to move the occlusion element to adjust a size of an orifice through which blood can flow based on the monitoring of the one or more outputs.

17. A method of treatment for reducing right atrial pressure of a heart of a subject and reducing intracranial pressure of the subject, the method comprising:
introducing an implantable device in a superior vena cava of the subject, the device comprising:
a first sensor configured to detect a first pressure indicative of a level of right atrial pressure;
a second sensor configured to detect a second pressure at a location upstream of the first sensor, wherein the second pressure is indicative of intracranial pressure;
a processing module electrically coupled to the first sensor and the second sensor and configured to monitor outputs from the first sensor and the second sensor;
actuating, based on the monitoring of the outputs, the device to modulate a flow of blood through the device.

18. The method of claim 17, further comprising: de-actuating the device to maintain or regain the flow of blood through the device.

19. The method of claim 17, further comprises modulating a volume of blood flowing from the into the right atrium to alternately decrease the right atrial pressure and decrease intracranial pressure.

20. The method of claim 17, wherein the device further comprises:
an expandable frame comprising an inflow end and an outflow end and a longitudinal axis extending therethrough; and
an occlusion element coupled to the frame, and
wherein the occlusion element is configured to radially collapse toward the longitudinal axis of the expandable frame and radially expand away from the longitudinal axis of the expandable frame to modulate the blood flow through the device.
